(19) United States
(12) Patent Application Publication
(10) Pub. No.: US 2026/0360372 A1
(43) Pub. Date: Nov. 12, 2026

(54) CODING CONCEPTS FOR CODING BIOPHYSIOLOGICAL AND/OR MULTI-CHANNEL DIGITAL SIGNALS

(71) Applicant: Fraunhofer-Gesellschaft zur Foerderung der angewandten Forschung e.V., Munich (DE)

(72) Inventors: Heiner KIRCHHOFFER, Berlin (DE); Christian HELMRICH, Berlin (DE); Christian RUDAT, Berlin (DE); Jonathan PFAFF, Berlin (DE); Heiko SCHWARZ, Berlin (DE); Detlev MARPE, Berlin (DE); Thomas WIEGAND, Berlin (DE)

(21) Appl. No.: 19/647,761

(22) Filed: Apr. 14, 2026

Related U.S. Application Data
(63) Continuation of application No. PCT/EP2024/078926, filed on Oct. 14, 2024.

(30) Foreign Application Priority Data
Oct. 15, 2023 (EP) 23203678.0

(51) Int. Cl. G10L 19/008; G10L 19/02; G10L 19/06

(52) U.S. Cl. CPC G10L 19/008; G10L 19/0212; G10L 19/06

(57) ABSTRACT

A decoder for decoding a biophysiological signal from a data stream is presented, wherein the decoder is configured to decode the biophysiological signal from the data stream using an audio decoding scheme. Further aspects are also described and are also applicable to other types of multi-channel digital signals.

### CROSS-REFERENCES TO RELATED APPLICATIONS

This application is a continuation of copending International Application No. PCT/EP2024/078926, filed Oct. 14, 2024, which is incorporated herein by reference in its entirety, and additionally claims priority from European Application No. EP 23203678.0, filed Oct. 15, 2023, which is incorporated herein by reference in its entirety.

Embodiments according to the invention relate to apparatuses and methods for coding a biophysiological signal and/or a multi-channel digital signal.

### BACKGROUND OF THE INVENTION

With progressing digitalization and use of data, the need for data compression increases, for the sake of transmission and wider accessibility. For example, in the field of medicine, exchange of medical data between medical staff may need to be facilitated, and observation and assessment of medical data may be performed by remote personal or programs (e.g., a medical person, computer program, and a neural network monitoring biophysiological signal of multiple people). In other technical fields, similar data compression and/or data transfer may be necessary for data, e.g., of seismic measurements, weather measurements, long term testing, and the like.

This is achieved by the subject matter of the independent claims of the present application.

Further embodiments according to the invention are defined by the subject matter of the dependent claims of the present application.

### SUMMARY

An embodiment may have a decoder for decoding a biophysiological signal from a data stream, configured to decode the biophysiological signal from the data stream using an audio decoding scheme.

Another embodiment may have an encoder for encoding a biophysiological signal into a data stream, configured to encode the biophysiological signal into the data stream using an audio coding scheme.

Another embodiment may have a decoder for decoding a multi-channel digital signal from a data stream, configured to decode channel grouping information from the data stream, and group K′ coded channels representing the multi-channel digital signal into Q sets of nᵢ coded channels, with nᵢ indicating the number of coded channels in set i, with Q≥i≥1, according to the channel grouping information, and decode at least one set j of the Q sets, for which nⱼ≥2, from the data stream using a multi-channel decoding scheme.

Another embodiment may have an encoder for encoding a multi-channel digital signal into a data stream, configured to encode channel grouping information into the data stream, using which K′ coded channels representing the multi-channel digital signal are grouped into Q sets of nᵢ coded channels, with nᵢ indicating the number of coded channels in set i, with Q≥i≥1, and encode at least one set j of the Q sets, for which nⱼ≥2, into the data stream using a multi-channel coding scheme.

Another embodiment may have a decoder for decoding a multi-channel digital signal from a data stream, configured to decode channel permutation information from the data stream, and decode N channels of the multi-channel digital signal from the data stream; and permute the K channels of the multi-channel digital signal according to channel permutation information so as to obtain the multi-channel digital signal.

Another embodiment may have an encoder for encoding a multi-channel digital signal into a data stream, configured to encode channel permutation information into the data stream, and encode N channels of the multi-channel digital signal into the data stream in a manner according to which the K channels of the multi-channel digital signal are to be permuted according to channel permutation information so as to obtain the multi-channel digital signal. Another embodiment may have a decoder for decoding a multi-channel digital signal from a data stream, configured to decode inter-channel delay information from the data stream, and decode K channels of the multi-channel digital signal from the data stream, and mutually delay the K channels according to the inter-channel delay information.

Another embodiment may have an encoder for encoding a multi-channel digital signal into a data stream, configured to encode inter-channel delay information into the data stream, and encode K channels of the multi-channel digital signal into the data stream in a state where the K channels are mutually delayed according to the inter-channel delay information. Another embodiment may have a decoder for decoding a multi-channel digital signal from a data stream, configured to decode K′ coded channels, which represent the multi-channel digital signal, from the data stream; and subject the K′ coded channels to a channel re-transformation which re-transforms co-aligned sample positions of the K′ coded channels so as to obtain K channels of the multi-channel digital signal.

Another embodiment may have an encoder for encoding a multi-channel digital signal into a data stream, configured to subject K channels of the multi-channel digital signal to a channel transformation which transforms co-aligned sample positions of the K channels so as to obtain K′ coded channels and encode the K′ coded channels which represent the multi-channel digital signal into the data stream.

Another embodiment may have a method for decoding a biophysiological signal from a data stream, the method comprising decoding the biophysiological signal from the data stream using an audio decoding scheme.

Another embodiment may have a method for decoding a multi-channel digital signal from a data stream, the method comprising decoding channel grouping information from the data stream, and grouping K′ coded channels representing the multi-channel digital signal into Q sets of nᵢ coded channels, with nᵢ indicating the number of coded channels in set i, with Q≥i≥1, according to the channel grouping information, and decoding at least one set j of the Q sets, for which nⱼ≥2, from the data stream using a multi-channel decoding scheme.

Another embodiment may have a method for decoding a multi-channel digital signal from a data stream, the method comprising decoding channel permutation information from the data stream, and decoding N channels of the multi-channel digital signal from the data stream; and permuting the K channels of the multi-channel digital signal according to channel permutation information so as to obtain the multi-channel digital signal.

Another embodiment may have a method for decoding a multi-channel digital signal from a data stream, the method comprising decoding inter-channel delay information from the data stream, and decoding K channels of the multi-channel digital signal from the data stream, and mutually delaying the K channels according to the inter-channel delay information.

Another embodiment may have a method for decoding a multi-channel digital signal from a data stream, the method comprising decoding K′ coded channels, which represent the multi-channel digital signal, from the data stream; and subjecting the K′ coded channels to a channel re-transformation which re-transforms co-aligned sample positions of the K′ coded channels so as to obtain K channels of the multi-channel digital signal.

Another embodiment may have a method for encoding a biophysiological signal into a data stream, the method comprising encoding the biophysiological signal into the data stream using an audio coding scheme.

Another embodiment may have a method for encoding a multi-channel digital signal into a data stream, the method comprising encoding channel grouping information into the data stream, using which K′ coded channels representing the multi-channel digital signal are grouped into Q sets of nᵢ coded channels, with nᵢ indicating the number of coded channels in set i, with Q≥i≥1, and encoding at least one set j of the Q sets, for which nⱼ≥2, into the data stream using a multi-channel coding scheme.

Another embodiment may have a method for encoding a multi-channel digital signal into a data stream, the method comprising encoding channel permutation information into the data stream, and encoding N channels of the multi-channel digital signal into the data stream in a manner according to which the K channels of the multi-channel digital signal are to be permuted according to channel permutation information so as to obtain the multi-channel digital signal.

Another embodiment may have a method for encoding a multi-channel digital signal into a data stream, the method comprising encoding inter-channel delay information into the data stream, and encoding K channels of the multi-channel digital signal into the data stream in a state where the K channels are mutually delayed according to the inter-channel delay information.

Another embodiment may have a method for encoding a multi-channel digital signal into a data stream, the method comprising subjecting K channels of the multi-channel digital signal to a channel transformation which transforms co-aligned sample positions of the K channels so as to obtain K′ coded channels and encoding the K′ coded channels which represent the multi-channel digital signal into the data stream.

Another embodiment may have a data stream generated using an encoding method according to the invention.

In accordance with a first aspect of the present invention, a decoder for decoding a biophysiological signal from a data stream is provided, wherein the decoder is configured to decode the biophysiological signal from the data stream using an audio decoding scheme.

It has been recognized that biophysiological signal can have periodic patterns (e.g., signals related to a heart activity and/or brain activity), wherein such periodic patterns (e.g., having distinct clustering in a frequency spectrum of biophysiological signal) is accessible to compression of an audio decoding scheme, which is commonly designed to utilize for compression the fact that audio signals usually exhibit periodic patterns. Therefore, the use of an audio decoding scheme is able to take advantage of characteristics of biophysiological signal for improved coding efficiency, despite not constituting a typical audio signal. Such a biophysiological signal can therefore be coded very efficiently, which allows storing larger amount of data (e.g., long term measurements) and easier transmission of the biophysiological signal. For example, the biophysiological signal can be transmitted to medical personal for remote observation and/or reviewing by one or more doctors. Furthermore, the biophysiological signal can be easier included in a digital file of a patient. This invention describes an apparatus for encoding (or decoding) arbitrary digital waveform data like, e.g. medical waveform data, into (or from) a bitstream. For example, let D be a sequence of digital waveform data with K channels where each channel Cᵢ with 0≤i<K comprises a sequence of digitally stored sample values, sampled at a predefined frequency fᵢ. Let c_{i,j} be the j-th digitally stored sample value in channel Cᵢ where index j may, for example, be associated with a time instant tⱼ=j/fᵢ. The digitally stored sample values may, for example, be integer values or floating point values.

Several existing codecs for encoding and decoding digital waveform signals may be useful for encoding and decoding of different types digital waveform data. For example, an audio codec like, e.g., the Advanced Audio Coding (AAC) may be suitable for the encoding and decoding of neurophysiological signals from electroencephalography (EEG), electrocardiograms (ECG), or electromyography (EMG). In other words, the digital waveforms may be biophysiological waveforms.

It has been recognized that similar advantages can be used in other technical that can also potentially exhibit periodic pattern such as seismic measurements, long time stress tests (e.g., exposure of a test object to vibrations), weather measurements (e.g., periodicity in wind speeds, light exposure, or water levels), and stock market charts. Further aspects can therefore be applied to coding a biophysiological signal or other digital multi-channel signals, as will be described in the following.

In accordance with a second aspect of the present invention, a decoder for decoding a multi-channel digital signal from a data stream is provided, wherein the decoder is configured to decode channel grouping information from the data stream, and group K′ coded channels representing the multi-channel digital signal into Q sets of nᵢ coded channels, with nᵢ indicating the number of coded channels in set i, with Q≥i≥1, according to the channel grouping information, and decode at least one set j of the Q sets, for which nⱼ≥2, from the data stream using a multi-channel decoding scheme.

It has been recognized that a grouping of the channels can provide a grouping that can improve coding. For example, a grouping may be provided between signals with or without (or not sufficient enough) periodic or waveform like patterns. Furthermore, a grouping can be provided between signals with similar characteristics (e.g., similar in one or more of amplitude, frequency, and offset). Therefore, different groups of signals may be coded differently (e.g., non-periodic data may be encoded without an audio coding scheme and/or without entropy coding) and/or different signals of the group may be used for improved coding, e.g., using inter-channel prediction and/or transformation with improved energy compression.

In accordance with a third aspect of the present invention, a decoder for decoding a multi-channel digital signal from a data stream is provided, wherein the decoder is configured to decode inter-channel delay information from the data stream, and decode K channels of the multi-channel digital signal from the data stream, and mutually delay the K channels according to the inter-channel delay information.

It has been recognized that multiple channels of a digital signal may have similar characteristics, which can be beneficial for coding techniques such as inter-channel prediction and transformations, wherein mutually delaying the K channels can improve a similarity of the channels and therefore the coding efficiency. Such offsets of delays between channels may, for example, occur due to the use of multiple sensors arranged at different locations (e.g., on a patient) and/or the use of different measuring sensor. Especially outside the field of audio coding, compression related techniques may not be considered as much, which includes the use of mutual delays between channels.

In accordance with a fourth aspect of the present invention, a decoder for decoding a multi-channel digital signal from a data stream may be provided, wherein the decoder is configured to decode K′ coded channels, which represent the multi-channel digital signal, from the data stream, and subject the K′ coded channels to a channel re-transformation which re-transforms co-aligned (e.g., temporally co-located) sample positions of the K′ coded channels so as to obtain K channels of the multi-channel digital signal.

It has been recognized that multiple channels may have some similarities, which (e.g., due to similar periodic patterns and/or amplitudes) can be exploited in a transformation, as transformations can be performed into domains that relate to and benefit from these similarities. For example, a transformation using waveform functions such as discrete cosine transform may result in clustering at certain frequency spectrums due to a periodic nature of the signals. This clustering can be used to represent to data in a more compact form (e.g., compression of energy).

In accordance with a fifth aspect of the present invention, a decoder for decoding a multi-channel digital signal from a data stream is provided, wherein the decoder is configured to decode channel permutation information from the data stream, and decode N channels of the multi-channel digital signal from the data stream, and permute the K channels of the multi-channel digital signal according to channel permutation information so as to obtain the multi-channel digital signal.

The permutation allows ordering the channels in pattern that can facilitate grouping and ordering of channels according to priority and/or purpose. Furthermore, the permutation may allow optimizing subsequent coding steps. For example, the permutation may allow arranging the channels in a correct order for application of a delay (e.g., in order of smallest to largest delay and/or in order to reduce the signalling of a mapping between delay and channel). Furthermore, a permutation may be determined that improves coding efficiency of a subsequent transformation (e.g., by ordering according to spectral distribution). The permutation can also supplement or compensate for a subsequent grouping. For example, the permutation may be performed for an optimized delay and/or transformation, wherein a grouping may subsequently improve coding efficiency.

In accordance with aspects of the present invention, encoders are provided with features that correspond to the decoders of the aspects described above. Furthermore, methods performed or performable by such decoders and encoders are provided.

The problem to be solved may be defined as how to improve a coding efficiency.

### BRIEF DESCRIPTION OF THE DRAWINGS

Embodiments of the present invention will be detailed subsequently referring to the appended drawings, in which:

FIG. 1a shows a schematic example of a decoder for decoding a biophysiological signal from a data stream;

FIG. 1b shows a schematic example of an encoder for encoding a biophysiological signal into a data stream;

FIG. 2 shows a schematic view of an audio decoding scheme that is used to decode a biophysiological signal from a data stream;

FIG. 3 shows a schematic view of an audio decoding scheme that is used to encode a biophysiological signal into a data stream;

FIG. 4 shows an encoder for encoding a multi-channel digital signal into a data stream as well as decoder for decoding the multi-channel digital signal from data stream;

FIG. 5a shows a schematic view of a decoder for decoding a multi-channel digital signal from a data stream;

FIG. 5b shows a schematic view of an encoder for encoding a multi-channel digital signal into a data stream;

FIG. 6a shows a shows a schematic view of a decoder for decoding a multi-channel digital signal from a data stream;

FIG. 6b shows a schematic view of an encoder for encoding a multi-channel digital signal into a data stream;

FIG. 7a shows a schematic example of a decoder for decoding a multi-channel digital signal from a data stream;

FIG. 7b shows a schematic example of an encoder for encoding a multi-channel digital signal into a data stream;

FIG. 8a shows a schematic view of a decoder for decoding a multi-channel digital signal from a data stream;

FIG. 8b shows a schematic view of an encoder for encoding a multi-channel digital signal into a data stream;

FIG. 9a shows a schematic view of the decoder of FIG. 8a, wherein a channel re-transformation involves a sequence of partial channel re-transformations;

FIG. 9b shows a schematic view of the encoder of FIG. 8b, wherein a channel transformation involves a sequence of partial channel transformations;

FIG. 10a shows a schematic view of the decoder of FIG. 8a, wherein the decoder is configured to decode inter-channel delay information from the data stream;

FIG. 10b shows a schematic view of the encoder of FIG. 8b wherein the multi-channel digital signal has N channels and the encoder is configured to encode inter-channel delay information into the data stream;

FIG. 11a shows the decoder of FIG. 1a, wherein the biophysiological signal is a multi-channel digital signal, and the decoding the multi-channel digital signal comprises grouping K′ coded channels; and

FIG. 11b shows the encoder of FIG. 1b, wherein the biophysiological signal is a multi-channel digital signal, and the encoding the multi-channel digital signal comprises grouping K′ coded channels.

### DETAILED DESCRIPTION OF THE INVENTION

Equal or equivalent elements or elements with equal or equivalent functionality are denoted in the following description by equal or equivalent reference numerals even if occurring in different figures.

In the following description, a plurality of details is set forth to provide a more throughout explanation of embodiments of the present invention. However, it will be apparent to those skilled in the art that embodiments of the present invention may be practiced without these specific details. In other instances, well-known structures and devices are shown in block diagram form rather than in detail in order to avoid obscuring embodiments of the present invention. In addition, features of the different embodiments described herein after may be combined with each other, unless specifically noted otherwise.

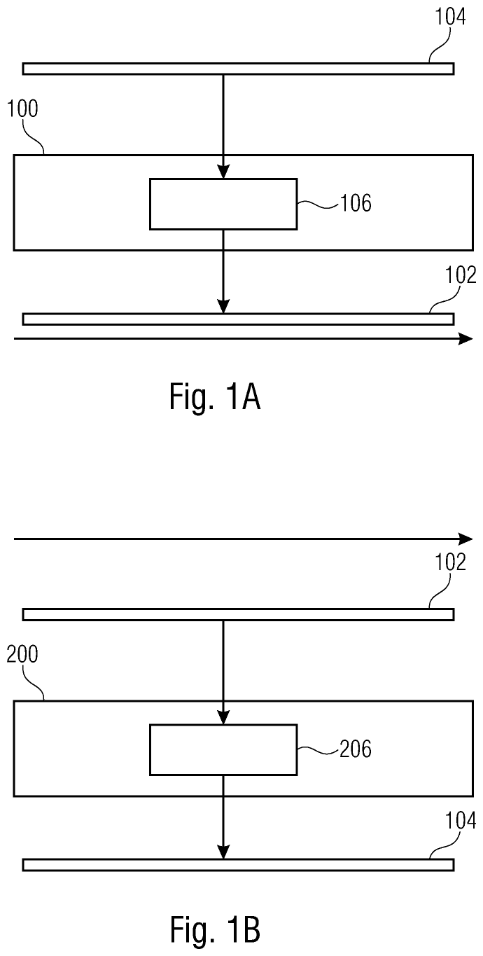

FIG. 1a shows a schematic example of a decoder 100 for decoding a biophysiological signal 102 from a data stream 104, configured to decode the biophysiological signal 102 from the data stream 104 using an audio decoding scheme 106.

FIG. 1b shows a schematic example of an encoder 200 for encoding a biophysiological signal 102 into a data stream 104, configured to encode the biophysiological signal 102 into the data stream 104 using an audio coding scheme 206.

It is noted that the decoded biophysiological signal 102 may be identical to the encoded biophysiological signal 102 encoded by the encoder 200, for example in the case of lossless coding. However, the biophysiological signal 102 and the encoded biophysiological signal 102 may be different, for example, due to a lossy compression or other modifications (e.g., change of sampling rate or amplitude during decoding). Similarly, the data streams 104 in FIGS. 1a and 1b may be identical (e.g., at least in terms of payload) or may be different (e.g., due to package loss and/or inclusion of transmission related signaling). The audio coding schemes 106 and 206 may be identical (e.g., wherein the encoder 200 is capable of performing an encoding aspect of the audio coding scheme 106/206 and the decoder 100 is capable of performing a decoding aspect of the audio coding scheme 106/206) or the audio coding scheme 106 comprises a decoding aspect that allows decoding a data stream 104 encoded by an encoding aspect of the audio coding scheme 206 (e.g., such that the audio coding schemes 106 and 206 do not necessarily have to be identical as long as a compatibility between decoder 100 and encoder 200 is provided).

The biophysiological signal 102 may be of an electrogram, e.g., obtained by at least one of Electrocardiogramcephalography, and Electromyography, e.g., medical waveform data, e.g., bio-physiological waveform data, e.g., bio-medical waveform data. The biophysiological signal 102 may be representative of a heart's electrical activity and/or movements (e.g., a heartbeat of a person). Alternatively or additionally, the biophysiological signal 102 may be representative of a brain's electrical activity. The biophysiological signal 102 may be or may be indicative of a voltage determined by electrodes (e.g., placeable or placed on a person's skin). The biophysiological signal 102 may be represented by an amplitude (e.g., voltage) over time. The biophysiological signal 102 may comprise a plurality of samples (e.g., each indicating an amplitude value) wherein the samples are arranged in a chronological order (which may or may not differ from a coding order), for example at a fixed or variable sampling rate (e.g., between 10Hz and 20kHz).

The biophysiological signal 102 may have a single channel or may have multiple channels. The biophysiological signal 102 may comprise a channel for each of an electrode configured to determine an electrical voltage (e.g., ten channels for voltages determined by ten electrodes).

The data stream 104 may be defined by the biophysiological signal 102 only or may comprise further data. For example, the data stream 104 may comprise data (e.g., one or more syntax elements) that indicates one or more of a type of data (e.g., whether the data stream 104 relates to a biophysiological signal or an audio signal) a type of biophysiological signal (e.g., Electrocardiogram Electroencephalography), a type of electrode, and patient information (e.g., one or more of age, name, gender, and insurance).

The audio decoding scheme 106, 206 may be configured to perform lossy or lossless compression of a plurality of samples (e.g., of an amplitude of an audio signal and/or the biophysiological signal 102). As will be discussed further below, the audio decoding scheme 106 may be capable of predictive coding and/or performing transformations of samples. In coding and decoding, the encoder 200 and decoder 100 may respectively be configured to treat samples (e.g., amplitude at time increments) of the biophysiological signal 102 as if they were samples (e.g., amplitude at time increments) of an audio signal. As will be described further below, the encoder 200 and decoder 100 may respectively be configured to disable the use of audiospecific techniques (e.g., psychoacoustic optimizations), e.g., that are related to the perception limits of the human ear. The disabling may be triggered by one or more syntax elements in the data stream (e.g., a flag or syntax element that indicates the coding of a non-audio data and/or coding of a biophysiological signal) and/or by a predetermined setting (e.g., when loading a coding program on a medical device or computer).

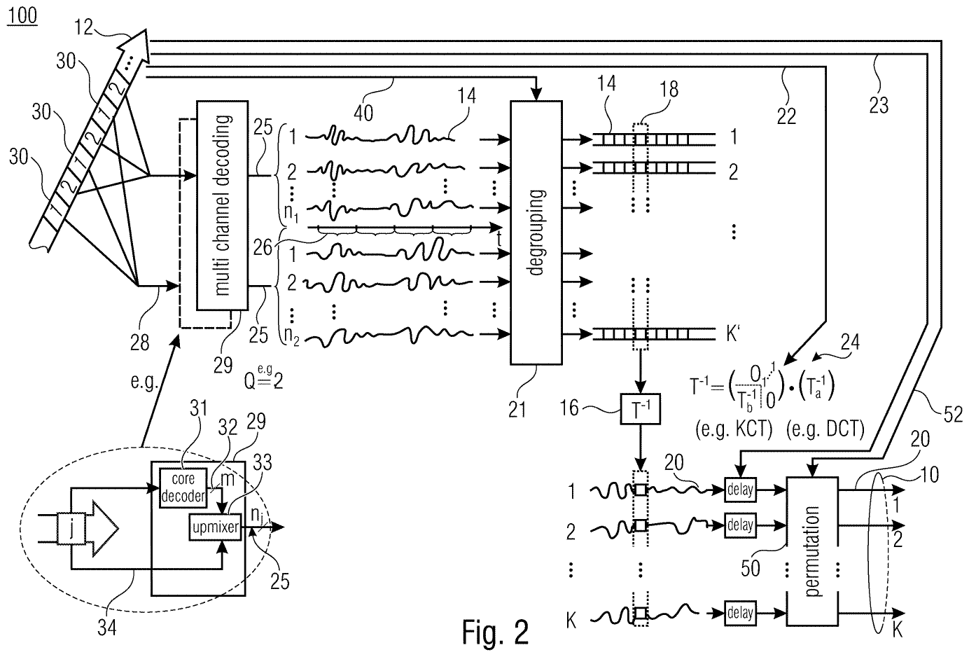

In the following, for the sake of conciseness, a combination of a plurality of optional features of the decoder 100 will be described with reference to a FIG. 2. However, it is noted that any feature described in FIG. 2 can be provided individually or in any combination with any other features described in FIG. 2 or the entire disclosure herein (e.g., with any of the aspects one to five described above in section 1.1, e.g., with any of the FIGS. 1a, 1b, or 3 to 11b). Furthermore, any feature disclosed with reference to the decoder 100 may be provided in a corresponding manner with the encoder 200.

FIG. 2 shows a schematic view of an audio decoding scheme that is used to decode a biophysiological signal 10 from a data stream 12. Any decoder 100 disclosed herein may be configured to use at least a portion of the audio decoding scheme (and vice versa any encoder 200).

The audio decoding scheme may involve (e.g. in a core decoder 31 that is part of the decoder 100) transform based audio decoding including deriving from the data stream 12 scale factors and transform coefficients, spectrally shaping the transform coefficients using the scale factors to obtain shaped spectra, re-transforming the shaped spectra to obtain audio frame signals and subjecting the audio frame signals to an overlap-add process (e.g., wherein the re-transformation is linear), and/or linear predictive coding (LPC) audio decoding including deriving from the data stream LPC coefficients and information on a residual signal and subjecting the residual signal to LPC synthesis by means of the LPC coefficients. It has been recognized that predictive coding can be particularly beneficial for decoding biophysiological signals, as such signals often exhibit a repeating behavior, which allows obtaining predictor with good accuracy.

The deriving of the scale factors from the data stream 12 may involve entropy decoding the scale factors from the data stream 12 or decoding LPC coefficients from the data stream 12 and converting the LPC coefficients into the scale factors. Entropy coding may involve context selection based on a previously decoded sample (e.g., bins thereof such as a significance and/or sign flag).

The biophysiological signal may be a multi-channel digital signal 10 (e.g., having two, three, four, or more channels), and the decoding the multi-channel digital signal from the data stream 12 using the audio decoding scheme comprises decoding K′ coded channels 14 representing the multi-channel digital signal from the data stream 12 by decoding n>1 coded channels with n≤K′ from the data stream 12 using the audio decoding scheme with the audio decoding scheme being a multi-channel audio decoding scheme 29 involving decoding 31 an m-channel downmix signal 32, with 0<m<n (e.g., a two-channel downmix signal 32), from the data stream 12 and deriving the n coded channels by upmixing 33 the m-channel downmix signal using side information 34 contained in the data stream. For example, the data stream 12 may comprise n=10 coded channels 14 of ten electrodes, wherein the ten channels 14 are downmixed to a two-channel downmix signal 32, wherein the side information 34 allows upmixing 33 the two-channel downmix signal in order to derive the ten coded channels 14.

Biophysiological signals may comprise redundancies such as similar periodicity, amplitude. Downmixing such signals may allow reducing such redundancies and subsequently improving coding efficiency.

The biophysiological signal may be a multi-channel digital signal, and the decoding the multi-channel digital signal from the data stream 12 using the audio decoding scheme, comprises grouping (e.g., which may also be termed degrouping or ungrouping, as the grouping may undo or degroup a grouping performed by a corresponding encoder) K′ coded channels 14 representing the multi-channel digital signal into Q sets 25 (for example Q=2 as shown in the example of FIG. 2) of nᵢ coded channels (e.g., a first set 25 with channels 1 to n₁ and a second set 25 with channels 1 to n₂ as shown in FIG. 2), with nᵢ indicating the number of coded channels in set i, with Q≥i≥1 (e.g., based on channel grouping information 40 decoded from the data stream 12), and decoding at least one set j of the Q sets 25 (e.g., j=1 or 2 in the example shown in FIG. 2), for which nⱼ≥2, from the data stream using the audio decoding scheme with the audio decoding scheme being a multi-channel audio decoding scheme involving decoding an m-channel downmix signal 32, with m<nⱼ from the data stream 12 and deriving the nⱼ coded channels 14 of set j by upmixing the m-channel downmix signal using side information 34 contained in the data stream 12. For example, the data stream 12 may comprise a two-channel downmix signal 32, wherein side information 34 contained in the data stream 12 allows deriving four channels (nⱼ=4) for a first set j=1 and deriving six channels (n₂=6) for a second set j=1. However, any other number of downmixed channels, upmixed channels and sets 25 may be provided. Channels 14 may be assigned to sets 25 according to one or more of signal similarity (e.g., similarity of one or more of amplitude, frequency, and temporal offset), coding dependency, signal type, and electrode type.

The K′ coded channels 14 may be grouped (or degrouped) based on signal similarities (e.g., one or more of amplitude, frequency, frequency spectrum, and phase). Alternatively or additionally, the K′ coded channels 14 may be grouped (or degrouped) based one or more priority criteria (e.g., according to spectral distribution, amplitude, or electrode). Different groups may be coded according to different priority (e.g., signals with higher frequencies may be transmitted last or not at all, e.g., in case of low transmission bandwidth).

Grouping (or degrouping) the K′ coded channels 14 allows gathering similar channels 14, which can improve coding efficiency during downmixing. Grouping (or degrouping) the K′ coded channels 14 according to a priority allows defining groups that have higher priority for signal decoding or signal displaying, and may subsequently be prioritized for transmission and/or selection of an error correction code.

The decoder 100 (or the audio decoding scheme) may be configured to subject the K′ coded channels of the multi-channel digital signal to a channel re-transformation 16 which re-transforms co-aligned (temporally co-located) sample positions 18 of the K′ coded channels 14 so as to obtain K (re-transformed) channels 20 of the multi-channel digital signal (e.g., with K=K′ or K different from K′) (e.g., wherein the channel re-transformation is linear).

Biophysiological signals may be approximated well by linear combinations of other channels, e.g., biophysiological signals may have a periodic pattern (e.g., a heart's electrical activity), which can be approximated well by linear combinations of wave functions, which may concentrate a signal energy. For example, the sample positions 18 of the K′ coded channels 14 may relate to a frequency distribution for wave functions, wherein the K channels 20 of the multi-channel digital signal obtained by re-transformation (e.g., using a fourier transformation such as discrete cosine transform (DCT) or the Karhunen-Loève transform (KLT)) may be (or form a basis for) the biophysiological signals.

The decoder 100 may be configured to derive the channel re-transformation from transformation information 22 in the data stream 12. For example, the transformation information 22 may be indicative (or define) a one or more transformation matrices (and/or its inverse or inverses). The transformation information 22 may define (or allow deriving) a Karhunen-Loève transform (e.g., its matrix parameters and/or dimensions).

The decoder 100 may be configured to update the channel re-transformation based on the transformation information 22 in the data stream (e.g. at each random access point, RAP) (so that different re-transformations are used before and after the updating). The transformation information 22 may comprise differential values and/or absolute values for updating the channel re-transformation.

The channel re-transformation may involves a sequence of partial channel re-transformations (see transformation 24 in FIG. 2). For example, twenty (e.g. K′=20) coded channels 14 may be provided in total, wherein a first one of the K channels 20 may be obtained by a re-transformation based on only a portion of the twenty co-aligned sample positions 18 (e.g., 16 out of 20 sample positions 18). A second one of the K channels 20 may be obtained by a re-transformation based on only another portion (or same portion as for the first channel) of the twenty co-aligned sample positions 18 (e.g., 16 out of 20 sample positions). A third one of the K channels 20 may obtained by a re-transformation based on all of the twenty co-aligned sample positions 18. The use of partial channel re-transformations can be combined with the use of full channel re-transformation (i.e. using samples of all K′ channels 14).

The partial channel re-transformations may be of different dimensions in terms of number of re-transformed channels 20 so that different coded channels 14 are affected by different subsets (wherein each subset may be a proper subset of the overall set encompassing all (re-)transformations or equal to the overall set), or different numbers or sub-sequences of (re-)transformations, out of the sequence of the partial channel re-transformations. The subsets may be different or identical to the sets 25.

The multi-channel decoding scheme may be designed to maximally cope with a maximum number MAX of coded channels 14 and the decoder 100 may be configured to group (or degroup) the K′ coded channels into the Q sets of nᵢ coded channels so that nᵢ≤MAX for each 0<j<Q+1. For example, the decoding scheme may be designed to maximally cope with a maximum number of six (e.g., MAX=8) coded channels 14 and the data stream 12 comprising 30 channels 14 (e.g., K′=30), wherein the decoder 100 may be configured to group the 30 channels 14 into four sets 25, e.g., three sets 25 having eight channels 14 each and a fourth set 25 having six channels 14. The maximum number MAX of coded channels 14 may be predefined. Alternatively, the maximum number MAX of coded channels 14 may be transmitted (e.g., once at the beginning of the data stream and/or in regular time intervals).

The decoder 100 may be configured to decode channel grouping information 40 from the data stream (e.g., as a basis for grouping the K′ coded channels) and perform the grouping using the channel grouping information. The channel grouping information 40 may indicate a value for a number of channels n in the Q sets, wherein, for example, the decoder 100 may be configured to group the channels into sets in according to a coding order. Alternatively or additionally, the channel grouping information 40 may indicate an assignment of one or more (or all) channels 14 to a corresponding set 25 (e.g., using a set ID and/or a set ID offset).

The channel grouping information 40 may comprise syntax elements indicating the number of coded channels n in the Q sets, and/or a sequential channel order among the K′ coded channels using which the K′ coded channels are grouped into the Q sets 25 so that each coded channels 14 of different ones of the Q sets are not interleaved along the sequential channel order. Therefore, the channel grouping information 40 may comprise explicitly signaled side information and/or implicit information conceived by a sequential channel order of the K′ coded channels.

The side information may comprises one or more of inter-channel coherence (ICC) data, channel level difference (CLD) data, and channel prediction coefficient (CPC) data.

The decoder 100 may be configured to decode inter-channel delay information 23 from the data stream, and mutually delay channels 20 (e.g., apply a delay to one or more of the channels 20) of the multi-channel digital signal according to the inter-channel delay information 23. The inter-channel delay information 23 may indicate (or define) a delay in time units (e.g., seconds or milliseconds) and/or sample units. The inter-channel delay information 23 may indicate an absolute delay, e.g., relative to an absolute or global time or relative to a pre-defined sample (e.g., a first sample, a first sample of a group of samples, a first sample of temporal block, or a first black of a temporal channel block). Alternatively or additionally, the inter-channel delay information 23 may indicate a relative delay, e.g., relative to a pre-defined channel (e.g., a first channel) of a group of channels or all channels. For example, the relative delay may be indicated relative to a first channel of the K channels 20 or relative to a first (in a temporal order) channel of the K channels that exceeds a threshold (e.g., a first channel that exceeds a pre-determined threshold, indicating that the other channels 20 are delayed).

For example, in FIG. 2, K channels 20 are obtained based on a re-transformation 16 (e.g., from a frequency domain into a time domain) of K′ channels 14, wherein inter-channel delay information 23 allows applying a delay, between the channels 20.

The biophysiological signal may comprise a plurality of signals that could potentially be compressed better (e.g., due to more similar predictions and/or better compressible sample distributions) if signal amplitudes are better aligned. For example, the biophysiological signal may comprise a plurality of voltage signals of a heart's electrical activity, wherein the signals may have similar periodicity but offset relative to each other in a temporal direction (e.g., due to electrodes being arranged at distances relative to the heart and/or the heart having an asynchronous distribution of its electrical distribution). For the sake of coding, compression may be improved by adding a delay that better aligns the channels 20 (e.g., temporally aligning a largest amplitude of channels 20). As a result, the delayed channels 20 are more similar to each other, which can reduce a complexity of sample values of when transformed (at the encoder) to channels 14, resulting potentially in a better compression. Furthermore, inter-channel prediction may be improved. After decoding, the delays can be reintroduced yielding a time delay or equal to (or closer to) the originally determined biophysiological signal. It is noted that the use of a delay applied to channels 20 does not necessarily require the use of a transformation and may be used also in case that the biophysiological signal is coded (and transmitted) without transformation (e.g., maintaining the signal in a time-domain without changing to a frequency domain).

The decoder 100 may be configured to permute (see reference sign 50 in FIG. 2) channels 20 of the multi-channel digital signal 10 according to channel permutation information 52 signaled in the data stream 12 so as to obtain the multi-channel digital signal 10. The channel permutation information 52 may indicate a mapping between pre-permutated channels 20 and permutated channels 20. The channel permutation information 52 may indicate an offset between channels 20. Alternatively or additionally, the decoder 100 may be configured to permute channels 20 of the multi-channel digital signal according to the channel grouping information 40. The channel permutation information 52 and the channel grouping information 40 may be transmitted at the beginning of the data stream and/or in the same (e.g., periodic) pattern.

The decoder 100 may be configured to decode each set of the Q sets 25 from a substream 28 of the data stream which may be associated with the respective set 25 (e.g., in two substreams 28 for the two sets 25 shown in FIG. 2) and in units of temporal intervals 26 (e.g., defining a pre-determined time interval such as 16, 32, 64, or 128 ms or a pre-determined amount of data, e.g., dependent on package payload) so that each substream 28 is formed by a sequence of substream portions 30 (see reference sign 30 in FIG. 2 for first substream portions with label “1”) having consecutive temporal intervals of the set 25 associated with the respective substream 28 encoded thereinto, and the substream portions 30 of the substreams 12 are mutually interleaved so that substream portions 30 whose temporal interval overlaps are immediately consecutive in the data stream 12 and precede substream portions 30 whose temporal interval temporally follows.

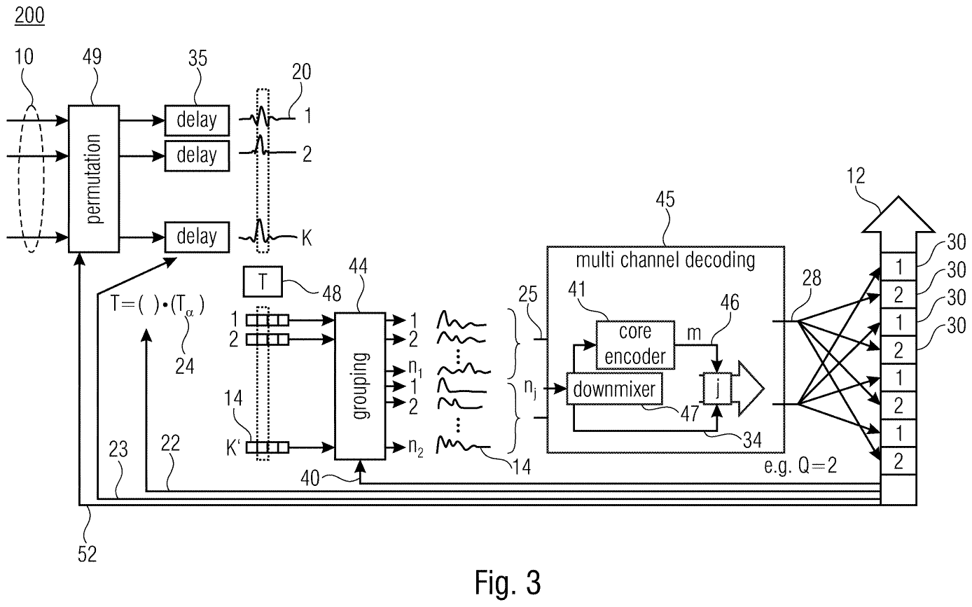

FIG. 3 shows a schematic view of an audio decoding scheme that is used to encode a biophysiological signal 10 into a data stream 12. Any encoder 200 disclosed herein may be configured to use at least a portion of the audio encoding scheme. The disclosure herein relating to any encoder is applicable to (or encompasses) a corresponding decoder 100 capable of decoding a data stream encoded by such an encoder 200.

The encoder 200 is configured to encode the biophysiological signal 10 into the data stream 12 using an audio coding scheme.

The audio coding scheme may involve (e.g. in a core encoder 41 that may be part of the encoder 200) transform based audio coding including inserting into the data stream 12 scale factors and transform coefficients, wherein the scale factors are for spectrally shaping the transform coefficients to obtain shaped spectra a re-transformation of which to obtain audio frame signals with subjecting the audio frame signals to an overlap-add process yields a reconstruction, or linear predictive coding (LPC) audio coding including inserting into the data stream LPC coefficients and information on a residual signal so that subjecting the residual signal to LPC synthesis by means of the LPC coefficients yields a reconstruction. The audio encoding scheme may involve the inserting of the scale factors into the data stream 12 and may involve entropy encoding the scale factors into the data stream or encoding LPC coefficients into the data stream that allow converting the LPC coefficients into the scale factors.

The biophysiological signal may be a multi-channel digital signal 10, and the encoding the multi-channel digital signal 10 into the data stream 12 using the audio encoding scheme, may comprise encoding K′ coded channels 14 representing the multi-channel digital signal 10 into the data stream 12 by encoding n>1 coded channels 14 with n≤K′ into the data stream 12 using the audio encoding scheme with the audio encoding scheme being a multi-channel audio encoding scheme 45 involving encoding 41 an m-channel upmix signal 46 (which may correspond to m-channel downmix signal 32), with 0<m<n, into the data stream 12 from which the n coded channels 14 are derivable by downmixing 47 the m-channel upmix signal 46 using side information 34 encoded into the data stream 12.

The biophysiological signal may be a multi-channel digital signal 10, and the encoding the multi-channel digital signal 10 into the data stream 12 using the audio encoding scheme may comprise grouping (see reference sign 44 in FIG. 3) K′ coded channels 14 representing the multi-channel digital signal into Q sets of nᵢ coded channels 14, with nᵢ indicating the number of coded channels in set i, with Q≥i≥1 (e.g., wherein the grouping may be derivable based on channel grouping information 40 encoded into the data stream 12), and encoding at least one set j of the Q sets, for which nⱼ≥2, into the data stream 12 using the audio encoding scheme with the audio encoding scheme being a multi-channel audio encoding 45 scheme involving encoding an m-channel downmix signal 46 (which may correspond to m-channel downmix signal 32), with m<nⱼ, into the data stream which allows deriving the nⱼ coded channels 14 of set j by upmixing the m-channel downmix signal 46 using the side information 34 contained in the data stream 12.

The encoder 200 may be configured to subject K channels 20 of the multi-channel digital signal 10 to a channel transformation 48 (e.g., an inverse transformation of re-transformation 16, e.g., wherein a combination of the channel transformation 48 and the re-transformation 16 result in a unity function) which transforms co-aligned (temporally co-located) sample positions of the K channels 20 so as to obtain the K′ coded channels 14 of the multi-channel digital signal 10 (e.g., with K=K′ or K different from K′, e.g., wherein the channel transformation is linear).

The encoder 200 may be configured to include transformation information 22 in the data stream 12 from which a channel re-transformation (e.g., channel re-transformation 16) corresponding to the channel transformation 48 is derivable.

The encoder 200 may be configured to change the channel transformation 48 and signal an update of the transformation information in the data stream accordingly (e.g. at each RAP, so that different transformations are or can be used before and after the updating).

The channel transformation 48 may involve a sequence of partial channel transformations (e.g., see reference sign 51 in FIG. 3).

The partial channel transformations 51 may be of different dimensions in terms of number of transformed channels 14 so that different coded channels 20 are affected by different subsets (wherein each subset may be a proper subset of the overall set encompassing all (re-)transformations or equal to the overall set), or different numbers or sub-sequences of (re-)transformations, out of the sequence of the partial channel transformations.

The multi-channel encoding scheme may be designed to maximally cope with a maximum number MAX of coded channels and the encoder 200 may be configured to group the K′ coded channels 14 representing the multi-channel digital signal 10 into the Q sets of nᵢ coded channels 14 so that nᵢ≤MAX for each 0<i<Q+1.

The encoder 200 may be configured to encode channel grouping information 40 into the data stream 12 (e.g., that allows deriving the grouping 44 of the K′ coded channels 14) which is indicative of the grouping 44.

The channel grouping information 40 may comprise syntax elements indicating the number of coded channels nᵢ in the Q sets (e.g., four channels 14 in a first set and eight channels 14 in a second set), and/or a sequential channel order among the K′ coded channels 14 using which the K′ coded channels 14 are grouped into the Q sets (e.g., by assigning coded channels 14 an ID of a set and/or providing a mapping between coded channels 14 and the sets 25) so that each coded channels 14 of different ones of the Q sets 25 are not interleaved along the sequential channel order.

The side information 34 may comprises one or more of ICC (inter-channel coherence) data, CLD (channel level difference) data, and CPC (channel prediction coefficient) data.

The encoder 200 may be configured to encode inter-channel delay information 23 into the data stream 12, and to encode the K channels 20 into the data stream 12 in a state where the K channels 20 are mutually delayed (see reference sign 35) according to the inter-channel delay information 23.

The encoder 200 may be configured to include channel permutation information 52 signaled in the data stream 12 and permute the channels 20 of the multi-channel digital signal 10 so as to encode the channels 20 of the multi-channel digital signal 10 in a permuted state.

The channel permutation information 52 may describe a permutation (see reference sign 49) between the channels 20 of the multi-channel digital signal 10 as encoded into the data stream 12 and a predetermined representation of the multi-channel digital signal 10.

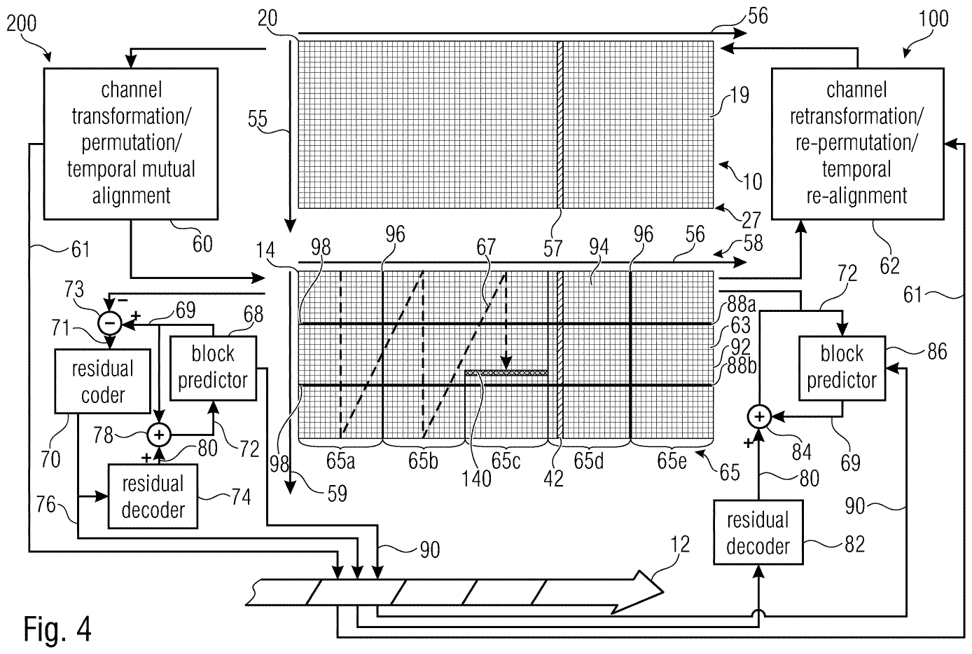

The above description is extended in the following by the presentation of further embodiments. Before this, however, the description proceeds with a presentation of a possible framework or codec into which the embodiments described above as well as the embodiments described further below may be built into. Many details described in this framework are, however, optional when being combined with any of the above or subsequently described embodiments. To be more precise, the framework is described with respect to FIG. 4 which shows an encoder 200 for encoding a multi-channel digital signal 10 into a data stream 12 as well as decoder 100 for decoding the multi-channel digital signal 10 from data stream 12. This description of FIG. 4 shall be seen as a presentation of new embodiments of the present application which result when combining any of the embodiments described above or any of the embodiments described subsequently is combined with the decoder 100 or encoder 200 of FIG. 4 either by adopting all details/functionalities described with respect to FIG. 4 or with leaving-out some of the details/functionalities described with respect to FIG. 4. Sometimes such “optional” features of FIG. 4 are explicitly identified as being optional with respect to the combination of the previously and subsequently described embodiments, but the just-mentioned possible combinations of the previously/subsequently explained embodiments with the description of FIG. 4 shall not be restricted to the these explicitly identified variations of FIG. 4 in terms of leaving-out certain features.

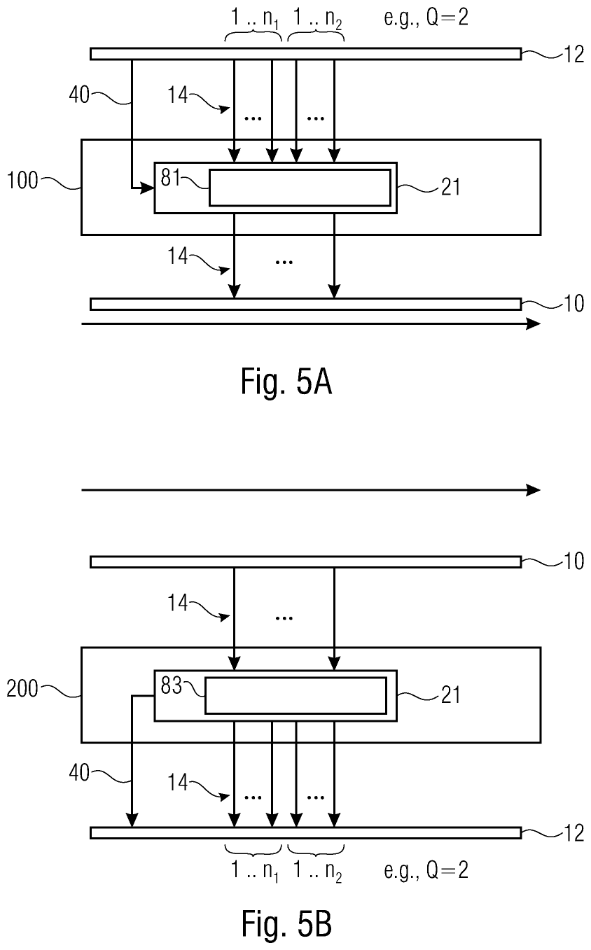

In FIG. 4, the multi-channel digital signal 10 is illustrated by way of an array of samples with the samples being illustrated as small squares 19 (which may correspond to sample positions 18 of FIGS. 2 and 3). Each line/row corresponds to a certain channel (e.g., channels 20 of FIGS. 2 and 3, e.g., with or without one or more of a transformation, permutation and temporal alignment) of the multi-channel digital signal 10. Each channel of signal 10 may have associated therewith a respective channel ID and FIG. 4 shows these channels as being ordered according to their channel ID along vertical axis 55 which, thus, corresponds to a “source” channel axis 55. The horizontal axis 56 corresponds to time so that samples 19 forming one column, or being horizontally aligned, are samples belonging to one common time instant. Such set/column of temporally co-located samples 19 is illustrated in FIG. 4 at 57. In the example of FIG. 5, the multi-channel digital signal 10 has 32 channels (along the vertical axis 55) each with 80 samples 19 (along the horizontal axis 56), resulting in an overall 32×80=2560 samples 19 (in an original domain 27). However, any other number of channels 20 and number of samples 19 may be used instead.

Each channel 20, thus, forms a digital time-varying signal or time/amplitude or time-to-amplitude signal (e.g., with 80 values for each channel 20 in the depicted time segment of FIG. 4). The multi-channel digital signal 10 might have been obtained by at least one of Electrocardiography Electroencephalography, Electromyography or seismic measurement. Differently speaking, the multi-channel digital signal might be a bio-physiological waveform data such as an electroencephalography (EEG) signal, an electrocardiogram (ECG), or an electromyography (EMG) signal, or seismic waveform data. However, each channel/signal might alternatively be another sort of waveform signal data such as scalar media data such as an audio signal and the signal 10 might be a multi-channel audio signal.

FIG. 4 illustrates the option according to which signal 10 is not coded directly, i.e., in the original domain 27, but in a so-called “coded domain” 58 which might differ from the original domain 27 by one or more of 1) channel transformation (e.g., channel transformation 48 shown in FIGS. 3), 2) channel permutation (e.g., permutation 49 shown in FIG. 3), and 3) temporal mutual channel alignment (e.g., delay 35 shown in FIG. 3). The channel transformation, if applied, transforms, per sample time instant, a set or column 57 of samples from domain 27 to domain 58. Thus, in domain 58, the sample pitch and the time axis is the same as in domain 27, but the meaning of the channels is different, i.e., the “source” channels of domain 27 become transformed channels in domain 58. Accordingly, the vertical axis in FIG. 4 for domain 58 is denoted as 59. Note that the channel transformation might leave the number of channels unchanged so that there is the same number of channels in domain 27 as well as domain 58 (e.g., K′=K, resulting also in 32×80=2560 samples 63), but different approaches are also possible (e.g., K′≠K). Generally, the channel transformation would aim at reducing redundancy and trying to condense the channels' energy onto a fewer number of channels in domain 58. As said, the channel transformation is optional. Accordingly, in general terms, the channels in domain 58 are called “coded channels” (which may correspond to channels 14 of FIGS. 2 and 3 are therefore referenced in the following with reference sign 14) in order to distinguish them from the “original” or “source” channels 20 of digital signal 10 in domain 27. The permutation is also optional and may be used in combination with, or without, the channel transformation. If used in combination with the channel transformation, the permutation may be performed prior (e.g., as shown in FIG. 3) to and/or or subsequent to the channel transformation in order to permute/sort the source channels 20 prior to transformation and the coded channels subsequent to the channel transformation. The channel transformation might be a discrete cosine transform (DCT), discrete sine transform (DST), fast Fourier transform (FFT) or any other transformation. The temporal mutual alignment is also optional and might be seen as a constant temporal alignment between the source channels 20 or the coded channels 14.

The module in encoder 200 performing the one or more of channel transformation, channel permutation and temporal mutual alignment is indicated in FIG. 4 as block 60. Side information 61 might be used in order to signal information on one or more of the following: 1) The channel transformation used, 2) information on the permutation(s) among the source channels and/or coded channels and 3) information on the mutual temporal alignment/delays between the source channels or coded channels wherein the temporal mutual alignment might be restricted to full sample precision. The side information 61 may comprise one or more of transformation information 22, inter-channel delay information 23, side information 34, channel grouping information 40, channel permutation information 52 (e.g., as shown in FIGS. 2 and 3). A corresponding block 62 in decoder 100 performs the reverse step, i.e., performs one or more of: 1) a channel retransformation (e.g., channel re-transformation 16 in FIGS. 2), 2) a re-permutation (e.g., permutation 50 in FIG. 2) of the source channels 20 and/or coded channels 14, and 3) a temporal re-alignment of the source channels or coded channels (e.g., delay 35 in FIG. 3). Note, that if no channel transformation takes place, the coded channels 14 are, in fact, equal to the source channels 20 except for (optionally) being temporally mutually aligned and/or being differently sorted due to permutation. Block 62 might be controlled by the before-mentioned side information 61.

Thus, the “actual coding” relates to the coded channels 14 in domain 58. In the coded domain 58, the coded channels 14 are depicted in FIG. 4 as lines or rows of samples 63, each extending along time axis (or horizontal axis) 56, the coded channels being depicted one on top of the other along coded channel axis 32—potentially ordered according to a coded channel ID the have associated therewith—so as to result into an array of samples 63 (which may correspond to samples of channels 20 in FIGS. 2 and 3). Again, although FIG. 4 depicts the case that the number of source channels equals the number of coded channels (wherein 32 source channels 20 and coded channels 14 are depicted in the example of FIG. 4), the number might be different. Further, if channel transformation is used, while there is no longer a clear association between source channels on the one hand and coded channels on the other hand, the temporal association remains: For each temporally co-located samples 57, there is a corresponding temporally co-located set 42 of samples 63 of the coded channels, wherein the set 42 in domain 58 is a column and might be a set of horizontally mutually offset samples in case of, and according to, the mutual temporal alignment, if applied. In case of FIG. 4, it has exemplarily been assumed that no such temporal alignment took place so that both sets 42 and 57 are pure columns in the time/channel representation. For example, the samples 63 of the set 42 may define (or indicate) coefficients of a transformation of a function along the vertical axis 55 that approximates samples 19 of set 57. Therefore, individual samples 63 of set 42 may not necessarily define (e.g., in a one-to-one mapping) individual samples 19 of set 57, but may define in combination said function along the vertical axis 55, for example, by re-transformation.

The actual coding is done in units of so-called temporal blocks 65. The term “temporal block” 65 is used so as to denote both a temporal portion of the multi-channel signal in domain 58, i.e., the set of coded channels, as well as a temporal portion of a certain coded channel. That is, for each temporal block 65, each coded channel has a temporal block such as block 140 (e.g., along a width of a temporal block parallel to the time axis 58) depicted for some (exemplary) temporal block 65c and same are mutually co-located. The coding is done sequentially along these blocks 140 (or temporal blocks 140 or temporal channel blocks 140), by following a coding/decoding order 67, which traverses the blocks 140 temporal block 65 by temporal block 65 with traversing temporally co-located blocks of the coded channels 14 along a channel order (e.g., corresponding to the order of the coded channels along axis 59). This coding/decoding order is illustrated in FIG. 4 at 67. That is, in case of temporal block 140 being the block currently to be coded/decoded, the previously decoded/encoded temporal blocks include all preceding temporal blocks of all coded channels (e.g., all blocks 140 of temporal blocks 65a, b) as well as the temporally co-located temporal blocks of coded channels preceding the coded channel 92 of temporal block 140 in channel order (e.g., all blocks 140 of the temporal block 65c that precede the currently coded block, e.g., all blocks 140 located above the currently coded block 140). These previously coded/decoded temporal blocks and their samples are illustrated in FIG. 4 by way of shading. In this regard, note that in FIG. 4, merely one temporal block 140 has been illustrated explicitly in order to reduce the complexity of FIG. 4. Thus, in the specification herein, reference sign 140 is sometimes used to indicate the currently encoded/decoded temporal block or to stand representatively for all temporal blocks (e.g., horizontal lines of samples 63 extending along a width of the blocks 65a, b, c). Further, as depicted in FIG. 4, the partitioning of signal 58 into temporal blocks 65 and 140, respectively, might be done in a manner so that these blocks 65 and 140, respectively, are non-overlapping (e.g., forming separate or disjoint sub-divisions of the signal 58).

The actual coding in units of the temporal blocks 140 is performed predictively. That is, the encoder 200 comprises a block predictor 68 which predicts the samples 63 of the currently coded temporal block 140, thereby yielding a prediction signal 69, and the prediction residual 71 formed by a subtraction between the actual sample values of temporal block 140 and the predicted samples of prediction signal 69 formed at a subtractor 73 is coded into the datastream 12 by residual coder 70. The residual coding in residual coder 70 may, or may not, involve a coding error by means of quantization. In any case, block predictor 68 uses the reconstructable version as being available by previously coded temporal blocks (e.g., previously coded blocks 140) in order to obtain the prediction signal 69. This reconstructable version 72 might be derived at encoder 200 by means of a residual decoder 74 which reverses potential coding loss, such as quantization by means of dequantization, manifesting itself in the residual signal 76 coded into datastream 12, and an adder 78 which sums-up prediction signal 69 and the reconstructable residual signal 80 as obtained by residual decoder 74.

The decoder 100 decodes the coded channels from data stream 12 in a corresponding manner, i.e., in units of the temporal blocks 65 or in temporal blocks 140, respectively, and using predictive decoding. To this end, the decoder 100 comprises a residual decoder 82, an adder 84 and a block predictor 86 which correspond to, and are mutually connected in the same manner as, elements 74, 78 and 68 of encoder 200. That is, the residual decoder 82 derives from the residual signal 76 in data stream 12 the reconstructable residual signal 80 for a currently decoded temporal block 140 which is then subject to addition with prediction signal 69 derived by block predictor 86 for temporal block 140 on the basis of the reconstructed version 72 of previously decoded temporal blocks at adder 84. The output of adder 84, thus, yields the reconstructed version 72 of the currently decoded temporal block 140 and becomes part of the pool of already decoded samples of previously decoded temporal blocks when the temporal blocks 140 of the coded channels are, in this manner, traversed along coding/decoding order 67 so as to reconstruct the coded channels in the coded domain 58.

In order to enable a high degree of random access capability, some of the temporal blocks 65 may be coded in a random access manner meaning that the coded channels 14 therein are coded independent from previous temporal blocks 65. Imagine, for instance, that temporal blocks 65b and 65e are random access temporal blocks. Then, none of the temporal channel blocks 140 in temporal block 65b as well as 65e would depend on any preceding temporal block 140 such as none temporal block within temporal block 65a forming a coding dependency basis for any temporal channel block 140 in temporal block 65b and none of the temporal channel blocks 140 within temporal blocks 65a to 65d forming a coding dependency basis for any of the temporal channel blocks 140 within temporal block 65e. Thus, in other words, coding dependencies are restricted so as to not reach-out beyond the border of a random access temporal block 65b and 65e towards any preceding temporal block 65. Such restriction might also hold for intermediate temporal blocks 65c to 65d between random access temporal blocks 65b and 65e in that same may not depend on any temporal block preceding the leading one among the random access temporal blocks 65b and 65e, here block 65b. Accordingly, leading temporal borders of the random access temporal blocks 65b and 65e are indicated by bold lines in FIG. 4.

Further, it might be that the coding of the coded channels 14 also interrupts or restricts inter-channel dependencies by coding one or more of the coded channels as random access coded channels so that coding dependencies of these random access coded channels, or even these random access coded channels and the intermediate coded channels therebetween, are restricted so as to not reach-out beyond such a random access coded channel toward any coded channel preceding that random access coded channel in channel order along axis 32. Two such random access coded channels 88a and 88b and their associated inter-channel dependency borders are illustrated in FIG. 4.

The block predictor 68 and 86 of encoder 200 and decoder 100, respectively, operate synchronously, i.e., they generate the same prediction signal 69 based on the previously encoded/decoded samples of previously encoded/decoded temporal blocks 140. On encoder side 200, the prediction for a certain temporal block 140 may be accompanied or determined by one or more prediction parameters. Same might be determined on encoder side based on a rate/distortion optimization. These prediction parameters 90 may be coded into data stream 12 and may be decoded from data stream 12 and used by block predictor 86 so as to perform the same prediction (e.g., prediction 69).

It might be that encoder 200 and decoder 100 support more than one prediction mode. For instance, encoder 200 and decoder 100 may support an intra prediction mode (which mode may also be called block-copy mode) according to which the currently encoded/decoded temporal block 140 is predicted based on the reconstructable sample values of previously encoded/decoded temporal blocks of the same coded channel to which the currently encoded/decoded temporal block 140 belongs, which is coded channel 92 in the example of FIG. 4. Additionally or alternatively, encoder 200 and decoder 100 may support an inter-prediction mode (which mode may also be called cross-channel prediction mode) according to which the currently encoded/decoded temporal block 140 is predicted based on the reconstructable sample values of previously encoded/decoded temporal blocks of coded channels preceding—in coding order 32—the coded channel 92 to which the currently encoded/decoded temporal block 140 belongs. Additionally or alternatively, there may be a mixed prediction mode according to which the prediction signal 69 is obtained by both, reconstructed/reconstructable sample values of previously encoded/decoded temporal blocks of coded channel 92 itself as well as reconstructed/reconstructable sample values of coded channels preceding coded channel 92 in channel order along axis 32. Beyond this, there may be temporal blocks 140 which are coded without any prediction at encoder 200 and decoded without any prediction at decoder 100 such as the first temporal blocks in the tiles 94 resulting from mutually separating the temporal blocks by means of the random access borders 96 on the one hand and the random access channel borders 98 on the other hand. This corresponds to the prediction signal 69 being set to zero and this may form an additional mode which could be called bypass mode. Additionally, or alternatively, there may be other modes such as ones deriving a DC predictor or linear function predictor for block 69 based on immediately preceding samples of block 140. The prediction parameters 90 may, thus, contain for a currently encoded/decoded temporal block 140 a prediction mode flag or prediction mode indicator indicating the prediction mode to be used for this currently encoded/decoded temporal block 140 and, optionally, one or more parameters parameterizing the prediction mode to be used for this currently encoded/decoded temporal block 140.

The aforementioned coding dependencies ought not to cross any of the borders 96 and 98 not only result from the just-described sample prediction capabilities of block predictor 68 and 86, respectively, but may optionally also result from other mechanisms such as parameter prediction according to which parameters such as the aforementioned prediction parameters 90 for a certain temporal block 140 are predicted based on coding parameters conveyed in the data stream 12 for any previous temporal block 140, or context derivation for context-adaptive entropy coding/decoding any coding parameter such as the prediction parameters 90 or any other side information such as side information 76 and 61 for temporal block 140 based on any coding parameter conveyed in the data stream 12 for any preceding temporal block.

That is, summarizing, the encoder 200 encodes the multi-channel signal 10 by transferring it into the coded domain 58 and then coding the coded channels 14 into data stream 12 in the just-described block-wise and predictive manner, wherein decoder 100 decodes the coded channels of coded domain 58 from data stream 12 and the corresponding block-wise and predictive manner with then gaining the multi-channel signal 10 in its original form 27 based on the coded channels 14 in coded domain 58 by means of segment 62. As said, the channel transformation (e.g., between domains 27 and 58) is optional and if not used, each sample 63 in the coded domain 58 may really correspond to one sample 19 in the original domain 27. If, further, the temporal mutual alignment is not used, each sample 63 exactly corresponds to a sample 19 in the original domain 27 at exactly the same time instant or, differently speaking, all temporally co-located samples 63 in coded domain 58 remain mutually temporally co-located in the original domain 27.

As mentioned before, FIG. 4 only represents a possible “framework” into which the previously described embodiments and the embodiments described subsequently may be built into. Many modifications may be performed with respect to FIG. 4, and some of these modifications might be mentioned in the subsequent description with respect to certain ones of the subsequently described embodiments, but these modifications shall then be treated as being also applicable with respect to other ones of the subsequently described embodiments. Further, as a final note, and without being treated as forming an exclusive list of further possible amendments of the description of FIG. 4, it shall be noted that the temporal blocks 65 might, other than illustrated in FIG. 4, vary in block length rather than being of a constant length as depicted in FIG. 4. For instance, encoder 200 may decide on the length of blocks 65 and signal the block length of blocks 65 (and the corresponding temporal blocks 140 of the coded channels) within data stream 12. Further, although not described before, it might be that residual coder and residual decoder 70 and 82 may use transform coding/decoding in order to convey the residual signal 76 in data stream 12. That is, the residual signal 80 may be conveyed in data stream 12 in transform domain by way of transform coefficients in residual signal 76. The transform domain might be a DCT, DST or an FFT. The transform may be non-overlapping, i.e. it may only transform residual signal 80 and its re-transform may only cover residual signal 76 within block 140, and/or may be non-windowed, i.e. the residual signal might be transformed without any transform window used to temporally shape the residual signal 80 before the transform. The transform domain, i.e. the transformation leading from time domain to transform domain which is used by the encoder to transform the prediction residual signal 80 to be coded und the corresponding re-transformation leading from transform domain to time domain which is used by the decoder to derive the prediction residual signal 80, or the transformation, might be selected from a set of available transforms including, for instance, one or more of 1) one or more DCTs, 2) one or more DSTs, and 3) an identity transform according to which the prediction residual signal 80 is coded into the data stream 12 in time domain directly. Some deblocking processing might be used to avoid blocking artifacts. If, alternatively, an overlapped transform is used, an overlap-add processing with re-transforms of immediately preceding/succeeding temporal blocks of the same coded channel might be used in order to completely reconstruct the current temporal block's 140 residual signal 76. Besides such transform-(residual)-coded blocks there might be temporal blocks 140 which, additionally or alternatively, are coded using, besides the block prediction by block predictor 68/86—which could be called a primary prediction-a secondary sample-wise prediction of the residual samples in residual block 71 such as by predicting a current sample's residual sample by means of already decoded values of preceding-in sample coding order-residual samples in block 71 or 80, with then correcting same by means of a secondary-prediction-residual sample decoded from the data stream 12. The secondary-prediction-residual samples for such a block may coded into the data stream 12 en block in a transform domain or sample-wise in time domain.

### Coding a Biophysiological Signal

This invention (e.g., the encoder 200 and decider 100) may convert a signal D (e.g., a biophysiological signal 102, e.g., the multi-channel digital signal 10) into one or more sub-signals (e.g., channels 20, 14 and/or sets 25) that are suitable for an efficient (lossless or lossy) encoding and decoding with a given target codec, like, e.g., Advanced Audio Coding (AAC). Note, that it may be advantageous to modify the encoder 200 and/or decoder 100 of the target codec in order to reflect the desired distortion behavior. For example, it may be advantageous to disable psychoacoustic optimizations in AAC, e.g., if the mean squared error shall be minimized for a lossy compression of signal D. For example, the encoder 200 and decoder 100 may be configured to use an audio coding scheme without psychoacoustic optimizations (e.g., without using one or more of an equal-loudness contour, auditory masking, and filtering and/or attenuating frequencies according to perception limits of the human ear) or using an audio coding scheme with one or more (or all) psychoacoustic optimizations disabled.

FIGS. 1a and 2a show a decoder 100 decoding a biophysiological signal 102 (e.g., multi-channel digital signal 10) from a data stream 104 and an encoder 200 for encoding a biophysiological signal 102 (e.g., multi-channel digital signal 10) into a data stream 104, wherein the examples of FIG. 2 to 4 show examples of such a decoder 100 and encoder 200 with further (optional) features such as channel-wise splitting with optional channel reordering (e.g., grouping 21, 44 as shown in FIGS. 2 and 3), inter-channel delay (e.g., delay 35 in FIG. 3 based on inter-channel delay information 23), linear combinations of coded channels (e.g., transformation and retransformtion 48, 16 shown in FIGS. 2 and 3), multi-layer linear combinations of coded channels (e.g., partial channel (re)-transformations 24, 51 in FIGS. 2 and 3). These further features can be particularly advantageous for coding of a biophysiological signal 102, for example, due to such signals potentially exhibiting a periodic characteristic, having delay between multiple signals (e.g., due to measurement or bodily functions themselves), and having similar signal patterns accessible to energy compression.

However, these features can also be beneficial by themselves, not necessarily in the context of coding a biophysiological signal. In the following, embodiments according to aspects will be described that are realized independent from the aspect of coding a biophysiological signal and other aspects. Therefore, any of the aspects may be realized individually (i.e. without other aspects) or in combination with one or more of the other aspects (e.g., in full or only a part thereof). Any disclosure below related to coding of a multi-channel digital signal may be applied to one or more of a biophysiological signal, a seismic measurement (e.g., seismic waveform data), stock market data, and weather data.

The sub-signals (e.g., channels 20, 14 and/or sets 25) may be encoded with the target codec and the resulting so-called sub-bitstreams (e.g., substream portions 30 as depicted in FIGS. 2 and 3) may be combined into a single bitstream (e.g., bit stream 12). For decoding of such a bitstream, the sub-bitstreams are decoded from the bitstream with the target codec and D (or a distorted version thereof) can be reconstructed.

Several different methods for converting D into one or more sub-signals and combining the associated sub-bitstreams into one single bitstream are presented in the following subsections.

### Channel-Wise Splitting with Optional Channel Reordering

In this invention, signal D (e.g., a media sample, e.g., an audio sample, a biophysiological signal 102, or a seismic measurement) can be converted to one or more digital waveform sub-signals so that each channel of the D (e.g., channels 14, 20) is contained in (at least) one digital waveform sub-signal. This technique may be used in order to reduce the number of channels of each sub-signal so far that it is supported by the target codec (e.g., grouping eight channels 14 into one set). Next, the sub-signals are encoded with the target codec and the resulting sub-bitstreams are stored in the bitstream (e.g., bit stream 12). For each sub-bitstream contained in the bitstream (e.g., substream 28 in bit stream 12, e.g., in form of substream portion 30), information about how the channels contained in the sub-bitstream are mapped to the channels of D may optionally be encoded into the bitstream (e.g., using channel grouping information 40). Note that this technique allows to create a sub-signal that contains an arbitrary subset of the channels of D in arbitrary order. In this way, channels that are similar (e.g., in form of one or more of amplitude, frequency, frequency spectrum, and offset) may be grouped together. Depending on the capabilities of the target codec, it may be beneficial for the coding efficiency if similar channels are encoded in the same sub-signal (e.g., similar channels 14 or 20 are grouped in the same set 25). This may, for example, be the case when the target codec employs a prediction from one channel to another, or sum-difference (also known as mid-side) joint-stereo coding of two channel signals with optional predictive coding of the difference signal using the (original or reconstructed) sum signal.

In a preferred embodiment, the K channels (e.g., K channels 20 or K′ channels 14) of D are split into Q sub-signals (e.g., two sets with Q=2 as shown in FIGS. 2 and 3) so that the channel indices associated with a sub-stream are consecutive and ascending (e.g., 1 to n₁ and 1 to n₂ as shown in FIGS. 2 and 3) and where Q is a predefined number (e.g., predefined to a number of two, three, four, or more). This allows for a particularly efficient signaling of the mapping between channels of D and the sub-signals. It may be sufficient to encode (or infer) for each sub-signal information (e.g., comprised by or formed by channel grouping information 40) about the number of contained channels (e.g., n₁ and n₂) and information about the channel index of the first channel of D that is contained in the sub-signal.

FIG. 5a shows a schematic view of a decoder 100 for decoding a multi-channel digital signal 10 from a data stream 12, configured to decode channel grouping information 40 from the data stream 12, and group (or ungroup or degroup) K′ coded channels 14 (or channels 20 as shown in FIG. 2) representing the multi-channel digital signal 10 into Q sets 25 (e.g., two sets in FIGS. 2 and 5a) of nᵢ coded channels, with nᵢ indicating the number of coded channels in set i, with Q≥i≥1, according to the channel grouping information 40, and decode at least one set j of the Q sets, for which nⱼ≥2, from the data stream 12 using a multi-channel decoding scheme 81.

The decoder 100 shown in FIG. 5a may additionally comprise any feature (e.g., in isolation or in any combination) of any decoder 100 disclosed herein, e.g., with reference to other figures (e.g., FIGS. 2 and 4).

The channel grouping information 40 may comprise syntax elements indicating the number of coded channels nᵢ in the Q sets 25, and/or a sequential channel order among the K′ coded channels 14 (or K channels 20, for example in case of no transformation) using which the K′ coded channels 14 are grouped into the Q sets 25 so that coded channels 14 of different ones of the Q sets are not interleaved along the sequential channel order and the one or more coded channels of one set immediately follow each other in the sequential channel order. For example, all channels 14 of a first set may be followed by all channels 14 of a second set, which may optionally be followed by all channels of further sets 25.

The decoder 100 may be configured to decode each set j of the Q sets 25, for which nⱼ≥2, from the data stream 12 using the multi-channel decoding scheme 71 (which may comprise or be part of any multi-channel decoding scheme disclosed herein, e.g., multi-channel audio decoding scheme 29).

Further features of the decoder 100 of FIG. 5a will in the following be described with reference to FIGS. 2 and 4, wherein it is noted that the use of an audio decoding scheme is optional. The description above for FIGS. 2 and 4 may partially or fully be applied to the decoder 100 shown in FIG. 5a.

The multi-channel decoding scheme 71 (e.g., multi-channel audio decoding scheme 29) may involve decoding an m-channel downmix signal 32, with m<nⱼ from the data stream 12 and deriving the nⱼ coded channels by upmixing 33 the m-channel downmix signal 32 using side information 34 contained in the data stream 12 to obtain the nⱼ coded channels. The side information 34 may comprise one or more of ICC (inter-channel coherence) data, CLD (channel level difference) data, and CPC (channel prediction coefficient) data.

The multi-channel decoding scheme 71 may be designed to maximally cope with a maximum number MAX of coded channels 14 and the decoder100 may be configured to group the K′ coded channels 14 representing the multi-channel digital signal 10 into the Q sets 25 of nᵢ coded channels so that nᵢ≤MAX for each 0<i<Q+1.

The decoder 100 may be configured to subject the K′ coded channels 14 representing the multi-channel digital signal 10 to a channel re-transformation 16 which re-transforms co-aligned (temporally co-located) sample positions of the K′ coded channels 14 so as to obtain K channels 20 of the multi-channel digital signal 10 (e.g., with K=K′ or K different from K', e.g., wherein the channel re-transformation is linear). The decoder 100 may be configured to derive the channel re-transformation 16 from transformation information 22 in the data stream 12. The decoder 100 may be configured to update the channel re-transformation 16 based on the transformation information 22 in the data stream 12 (e.g. at each RAP, e.g., so that different channel re-transformations are used before and after the updating). The channel re-transformation 16 may involve a sequence of partial channel re-transformations (e.g., see reference sign 24 in FIG. 2). The partial channel re-transformations 24 may be of different dimensions in terms of number of re-transformed channels 20 so that different coded channels 14 are affected by different subsets (wherein each subset may be a proper subset of the overall set encompassing all (re-)transformations or equal to the overall set), or different numbers or sub-sequences of (re-)transformations, out of the sequence of the partial channel re-transformations 24.

The multi-channel decoding scheme 71 may be or use a multi-channel audio decoding scheme (e.g., multi-channel audio decoding scheme 29). The multi-channel decoding scheme 29 may involve transform based decoding (e.g. see reference sign 31 in FIG. 2) including deriving from the data stream 12 scale factors and transform coefficients, spectrally shaping the transform coefficients using the scale factors to obtain shaped spectra, re-transforming the shaped spectra to obtain frame signals and subjecting the frame signals to an overlap-add process (e.g., wherein the re-transformation is linear), or linear predictive coding (LPC) decoding including deriving from the data stream 12 LPC coefficients and information on a residual signal and subjecting the residual signal to LPC synthesis by means of the LPC coefficients. The deriving of the scale factors from the data stream 12 may involve entropy decoding the scale factors from the data stream 12 or decoding LPC coefficients from the data stream 12 and converting the LPC coefficients into the scale factors.

The multi-channel digital signal 71 may have K channels 20 and the decoder 100 may be configured to decode inter-channel delay information 23 from the data stream 12, and mutually delay 35 the K channels 20 according to the inter-channel delay information 23. The multi-channel digital signal 10 may comprise (e.g., or is) a biophysiological signal (e.g., biophysiological signal 102). The decoder 100 may be configured to permute (e.g., see reference sign 50 in FIG. 2) the channels 20 of the multi-channel digital signal 10 according to channel permutation information 52 signaled in the data stream 12 so as to obtain the multi-channel digital signal 10. The channel permutation information 52 may describe a permutation of the channels 20 so as to result into a predetermined representation of the multi-channel digital signal 10.

FIG. 5b shows a schematic view of an encoder 200 for encoding a multi-channel digital signal 10 into a data stream 12, configured to encode channel grouping information into the data stream 12, using which K′ coded channels 14 representing the multi-channel digital signal are grouped into Q sets of nᵢ coded channels, with nᵢ indicating the number of coded channels in set i, with Q≥i≥1, and encode at least one set j of the Q sets, for which nⱼ≥2, into the data stream using a multi-channel coding scheme 83.

It is noted that the encoder 200 may be configured to determine the channel grouping information 40 by using rate distortion (R/D) testing and aiming at extremizing a R/D dependent measure, or by determining one or more measures for different settings of the channel grouping information 40 and selecting a setting out of the different settings which extremizes the one or more measures or a combined measure determined by the one or more measures. The multi-channel coding scheme 83 may be identical to the multi-channel coding scheme 81. The multi-channel coding scheme 81 and the multi-channel coding scheme 83 may be a decoding and encoding aspect of an overall multi-channel coding scheme. The multi-channel coding schemes 81, 83 may be any multi-channel coding schemes disclosed herein.

The encoder 200 may be configured to encode a data stream 12 decodable by the decoder 100 described with reference to FIG. 5a (or any other decoder 100 described herein). The encoder 200 may have any corresponding functionality and/or feature corresponding to functionalities and/or features described for the decoder 100 of FIG. 5a.

According to another aspect, a decoder is provided that is configured decode to channel permutation information as will be described in the following.

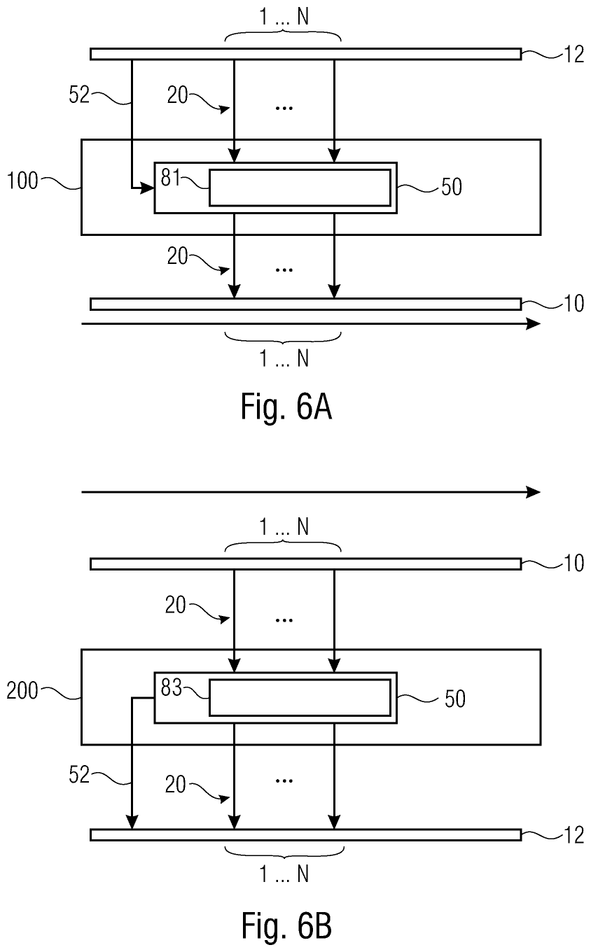

FIG. 6a shows a shows a schematic view of a decoder 100 for decoding a multi-channel digital signal 10 from a data stream 12, configured to decode channel permutation information 52 from the data stream 10, and decode N channels 20 of the multi-channel digital signal 10 from the data stream 12, and permute 50 the K channels 20 of the multi-channel digital signal 10 according to the channel permutation information 52 so as to obtain the multi-channel digital signal 10.

It is noted that the encoder 200 may be configured to determine the channel permutation information 52 by using rate distortion (R/D) testing and aiming at extremizing a R/D dependent measure, or by determining one or more measures for different settings of the channel permutation information and selecting a setting out of the different settings which extremizes the one or more measures or a combined measure determined by the one or more measures.

Further features of the decoder 100 will be described with reference to the examples shown in 2 and 4.

The channel permutation information 52 may describe a permutation 50 of the N channels 20 so as to result into a predetermined representation of the multi-channel digital signal 10. The decoder 100 may be configured to decode the N channels 20 of the multi-channel digital signal 10 from the data stream 12 using a multi-channel decoding scheme (e.g., any multi-channel decoding scheme 10 disclosed herein, e.g., the biophysiological signal 102). The decoder 100 may be configured to decode the N channels 20 of the multi-channel digital signal 10 from the data stream 12 by grouping K′ coded channels 14 representing the multi-channel digital signal 10 into Q sets 25 of nᵢ coded channels, with nᵢ indicating the number of coded channels in set i, with Q≥i≥1, and decode at least one set j of the Q sets, for which nⱼ≥2, from the data stream 12 using a multi-channel decoding scheme 81. The decoder 100 may be configured to decode each set j of the Q sets 25, for which nⱼ≥2, from the data stream 12 using a multi-channel decoding scheme 81 (which may be any multi-channel decoding scheme disclosed herein). The multi-channel decoding scheme 81 may involve decoding an m-channel downmix signal 32, with m<nⱼ from the data stream 12 and deriving the nⱼ coded channels by upmixing the m-channel downmix signal 32 using side information 34 contained in the data stream 12 to obtain the nⱼ coded channels 14. The side information 34 may comprise one or more of ICC (inter-channel coherence) data, CLD (channel level difference) data, and CPC (channel prediction coefficient) data.

The multi-channel decoding scheme 81 may be or use a multi-channel audio decoding scheme (e.g., any multi-channel audio decoding scheme disclosed herein). The multi-channel decoding scheme 81 may involve transform based decoding (e.g. see reference sign 31 in FIG. 2) including deriving from the data stream scale factors and transform coefficients, spectrally shaping the transform coefficients using the scale factors to obtain shaped spectra, re-transforming the shaped spectra to obtain frame signals and subjecting the frame signals to an overlap-add process (e.g., wherein the re-transformation is linear), or linear predictive coding (LPC) decoding including deriving from the data stream LPC coefficients and information on a residual signal and subjecting the residual signal to LPC synthesis by means of the LPC coefficients. The deriving of the scale factors from the data stream 12 may involve entropy decoding the scale factors from the data stream or decoding LPC coefficients from the data stream and converting the LPC coefficients into the scale factors. The multi-channel decoding scheme 81 may be designed to maximally cope with a maximum number MAX of coded channels and the decoder is configured to group the K′ coded channels representing the multi-channel digital signal into the Q sets of nᵢ coded channels so that nᵢ≤MAX for each 0<i<Q+1.

The decoder 100 may be configured to decode N channels 20 of the multi-channel digital signal 10 from the data stream 12 by decoding (see reference sign 29 in FIG. 2) K′ coded channels 14 representing the multi-channel digital signal 10 from the data stream 12, and subjecting the K′ coded channels 14 representing the multi-channel digital signal 10 to a channel re-transformation 16 which re-transforms co-aligned (temporally co-located) sample positions 18 of the K′ coded channels 14 so as to obtain the K channels 20 of the multi-channel digital signal 10 (e.g., with K=K′ or K different from K′, e.g., wherein the channel re-transformation is linear). The decoder 100 may be configured to derive the channel re-transformation 16 from transformation information 22 in the data stream 12. The decoder 100 may be configured to update the channel re-transformation 16 based on the transformation information 22 in the data stream 12 (e.g. at each RAP, e.g., so that different channel re-transformations are used before and after the updating). The channel re-transformation 16 may involve a sequence of partial channel re-transformations (e.g., see reference sign 24 in FIG. 2). The partial channel re-transformations 24 may be of different dimensions in terms of number of re-transformed channels 20 so that different coded channels 14 are affected by different subsets (wherein each subset may be a proper subset of the overall set encompassing all (re-)transformations or equal to the overall set), or different numbers or sub-sequences of (re-)transformations, out of the sequence of the partial channel re-transformations 24.

The decoder 100 may be configured to decode inter-channel delay information 23 from the data stream 12, and mutually delay 35 the K channels 20 according to the inter-channel delay information 23. The multi-channel digital signal 10 may comprise (e.g., or is) a biophysiological signal (e.g., any biophysiological signal disclosed herein).

FIG. 6b shows a schematic view of an encoder 200 for encoding a multi-channel digital signal 10 into a data stream 12, configured to encode channel permutation information 52 into the data stream 12, and encode N channels 20 of the multi-channel digital signal 10 into the data stream 12 in a manner according to which the K channels 20 of the multi-channel digital signal 10 are to be permuted 50 according to channel permutation information 52 so as to obtain the multi-channel digital signal 10.

The encoder 200 may have one or more additional features of any encoder 200 disclosed herein. The encoder 200 shown in FIG. 6b may be configured to encode a data stream 12 decodable by the decoder 100 shown in FIG. 6a. The encoder 200 may comprise any encoder side features that corresponding to features of the decoder 100 shown in FIG. 6a.

### Inter-Channel Delay

Particular types of digital waveform signals may reveal a high degree of correlation between channels. For example, spikes in an ECG recording are caused by the operation of the human heart (e.g., voltages spikes indicative of a heart's electrical activity). Therefore, it is likely that they occur in a similar way in different channels of an ECG recording at the almost the same instants in time. However, depending on the distances of the positions of the ECG electrodes to the human heart, there may exist a (e.g., constant) temporal delay between spikes in different channels. This may be a disadvantage for the target codec when predicting between channels.

In this invention, the sequence D (e.g., channels 14 or channels 20 as shown in FIGS. 2 and 3) can be converted to one or more digital waveform sub-signals so that the sequence of samples of each channel can be prefixed by predefined number of (artificially created) padding sample values (or any other form of delay such as fixed or variable time durations) which correspond to a delay. Information about the delay associated with a particular channel may be transmitted in the bitstream (e.g., bit stream 12). The decoder may use this delay information (e.g., inter-channel delay information 23) to remove the padding sample values (or any other form of delay, e.g., included by a corresponding encoder 200) after decoding and restore (the potentially distorted) sequence D with the correct inter-channel delays.

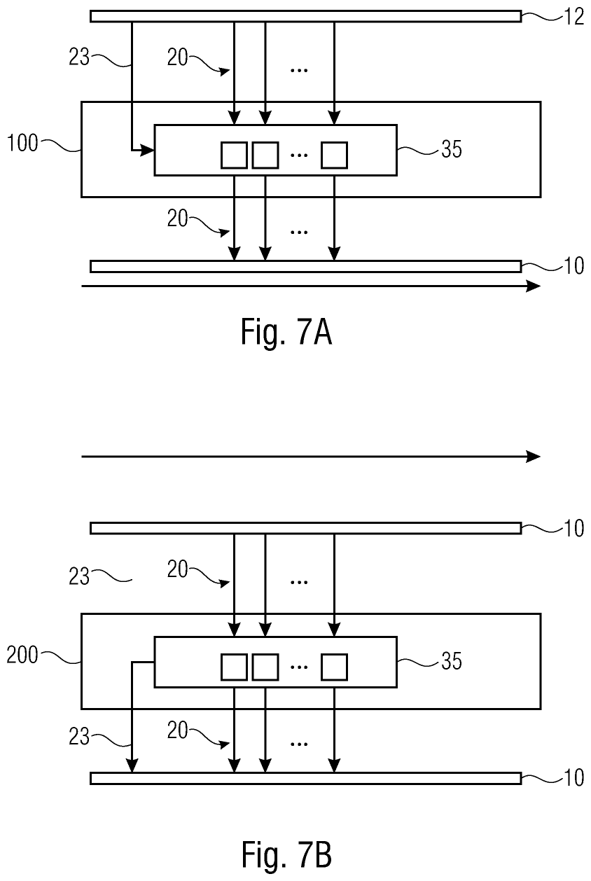

FIG. 7a shows a schematic example of a decoder 100 for decoding a multi-channel digital signal 10 (e.g., comprising or constituting a biophysiological signal) from a data stream 12, configured to decode inter-channel delay information 23 from the data stream 12, and decode K channels 20 of the multi-channel digital signal 10 from the data stream 12 (e.g. using inter-channel dependencies such as inter-channel prediction, M/S decoding), and mutually delay (see reference sign 35) the K channels 20 according to the inter-channel delay information 23.

The decoder 100 shown in FIG. 7a may additionally comprise any feature (e.g., in isolation or in any combination) of any decoder 100 disclosed herein, e.g., with reference to other figures (e.g., FIGS. 2 and 4). Further features of the decoder 100 of FIG. 7a will in the following be described with reference to FIGS. 2 and 4, wherein it is noted that the use of an audio decoding scheme is optional. The description above for FIGS. 2 and 4 may partially or fully be applied to the decoder 100 shown in FIG. 7a.

The decoder 100 may be configured to decode the K channels 20 using a multi-channel decoding scheme (e.g., a multi-channel audio decoding scheme and/or a spatial-object audio decoding scheme). The multi-channel decoding scheme (e.g., audio decoding scheme) may involve (e.g. see reference sign 31 in FIG. 2) transform based multi-channel (e.g., audio) decoding including deriving from the data stream 12 scale factors and transform coefficients, spectrally shaping the transform coefficients using the scale factors to obtain shaped spectra, re-transforming the shaped spectra to obtain frame signals and subjecting the frame signals to an overlap-add process (e.g., wherein the re-transformation is linear), or linear predictive coding (LPC) decoding including deriving from the data stream LPC coefficients and information on a residual signal and subjecting the residual signal to LPC synthesis by means of the LPC coefficients. The deriving of the scale factors from the data stream 12 may involve entropy decoding the scale factors from the data stream 12 or decoding LPC coefficients from the data stream and converting the LPC coefficients into the scale factors. The multi-channel digital signal may comprise (e.g., or is) a biophysiological signal.

The decoding the K channels from the data stream 12 may comprise decoding n>1 coded channels 14 representing the multi-channel digital signal 10 from the data stream 12 using a multi-channel decoding scheme 29 involving decoding 31 an m-channel downmix signal 32, with 0<m<n, from the data stream 12 and deriving the n coded channels by upmixing 33 the m-channel downmix signal using side information 34 contained in the data stream 12. The decoding the K channels 20 from the data stream may comprise grouping K′ coded channels 14 representing the multi-channel digital signal 10 into Q sets 25 of nᵢ coded channels, with nᵢ indicating the number of coded channels in set i, with Q≥i≥1 (e.g., based on channel grouping information 40 decoded from the data stream, e.g., with K=K′ or K different from K'), and decoding at least one set j of the Q sets 25, for which nⱼ≥2, from the data stream 12 using a multi-channel decoding scheme (such as an audio decoding scheme, e.g., the decoding scheme involving decoding an m-channel downmix signal 32, with m<nⱼ from the data stream 12 and deriving the nⱼ coded channels 14 of set j by upmixing the m-channel downmix signal using side information 34 contained in the data stream 12). The decoding the K channels 20 from the data stream 12 may comprise decoding n≤K′ coded channels 14 representing the multi-channel digital signal 10 from the data stream 12 using a multi-channel decoding scheme (such as an audio decoding scheme, e.g., the decoding scheme involving decoding an m-channel downmix signal 32, with m<n from the data stream and deriving the n coded channels 14 by upmixing the m-channel downmix signal using side information 34 contained in the data stream).

The decoder 100 may be configured to subject the K′ coded channels 14 of the multi-channel digital signal 10 to a channel re-transformation 16 which re-transforms co-aligned (temporally co-located) sample positions 18 of the K′ coded channels 14 so as to obtain the K channels 20 of the multi-channel digital signal 10 (e.g., wherein the channel re-transformation 16 is linear). The decoder 100 may be configured to derive the channel re-transformation 16 from transformation information 22 in the data stream 12. The decoder 100 may be configured to update the channel re-transformation 16 based on the transformation information 22 in the data stream 12 (e.g. at each RAP, e.g., so that different channel re-transformations are used before and after the updating. The channel re-transformation may involve a sequence of partial channel re-transformations (see reference sign 24 in FIG. 2). The partial channel re-transformations 24 may be of different dimensions in terms of number of re-transformed channels 20 so that different coded channels 14 are affected by different subsets (wherein each subset may be a proper subset of the overall set encompassing all (re-)transformations or equal to the overall set), or different numbers or sub-sequences of (re-)transformations, out of the sequence of the partial channel re-transformations.

The multi-channel decoding scheme may be designed to maximally cope with a maximum number MAX of coded channels 14 and the decoder is configured to group 21 the K′ coded channels 14 representing the multi-channel digital signal 10 into the Q sets 25 of nᵢ coded channels so that nᵢ≤MAX for each 0<i<Q+1.

The decoder 100 may be configured to decode channel grouping information 40 from the data stream 12 (e.g., as a basis for grouping the K′ coded channels 14) and perform the grouping 21 using the channel grouping information 40. The channel grouping information 21 may comprise syntax elements indicating the number of coded channels nᵢ in the Q sets 25, and/or a sequential channel order among the K′ coded channels 14 using which the K′ coded channels 14 are grouped into the Q sets 25 so that coded channels 14 of different ones of the Q sets are not interleaved along the sequential channel order and the one or more coded channels 14 of one set immediately follow each other in the sequential channel order.

The side information may comprise 21 one or more of ICC (inter-channel coherence) data, CLD (channel level difference) data, and CPC (channel prediction coefficient) data. The decoder 100 may be configured to permute 50 the K channels 20 of the multi-channel digital signal 10 according to channel permutation information 52 signaled in the data stream 12 so as to obtain the multi-channel digital signal 10. The channel permutation information 52 may describe a permutation of the N channels 20 obtained by the channel re-transformation 48 so as to result into a predetermined representation of the multi-channel digital signal 10.

FIG. 7b shows a schematic example of an encoder 200 for encoding a multi-channel digital signal 10 into a data stream 12, configured to encode inter-channel delay information 23 into the data stream 12, and encode K channels 20 of the multi-channel digital signal into the data stream 12 in a state where the K channels 20 are mutually delayed 35 according to the inter-channel delay information 23.

The encoder 200 shown in FIG. 7b may have one or more additional features of any encoder 200 disclosed herein. The encoder 200 shown in FIG. 7b may be configured to encode a data stream 12 decodable by the decoder 100 shown in FIG. 7a. The encoder 200 may comprise any encoder side features that corresponding to features of the decoder 100 shown in FIG. 7a.

It is noted that the delay 35 might be determined be the encoder 200 so as to, optionally and/or in case of the frame lengths for encoding the various coded channels 20 being different, balance the different audio frame border starting positions of the various channels 20 and so as to temporally mutually align the coded channels 20 so that same are easier or more efficiently to encode, such as using an optimization scheme which minimizes a mutual similarity measure between the channels 20. Note further, that, according to an example, all K channels 20 might already be equally sampled, i.e. in terms of sampling frequency and sampling delay/times, or that the encoder may re-sample the channel to encode so that this status is achieved. It may, however, also be that the grouping and/or the re-sampling is performed to the extent that this sampling adjustment is merely valid for channels within one set (e.g., sets 25), but not necessarily for channels belonging to different sets. Even further, sampling rate deviations in that one channel being sampled at a sampling rate which is an integer multiple of another channel, might be allowed, too. In even other embodiments, the encoder 200 may leave the channels 20 sampled as they are, irrespective of the mutual sampling times/frequencies difference.

### Linear Combinations of Decoded Channels

Particular types of digital waveform signals may contain channels (e.g., channels 20 and/or 14) that can be approximated well as linear combinations of other channels (or functions). Furthermore, a digital waveform signal can also be converted by employing a linear transform like, e.g., the discrete cosine transform (DCT) or the Karhunen-Loève transform (KLT), in order to concentrate the signal energy.

In this invention, the sequence D (e.g., multi-channel digital signal 10) can be converted to one or more digital waveform sub-signals in a way so that the decoder can reconstruct the channels of D by creating linear combinations (e.g., weighted sums) of the channels 14 as decoded from the sub-bitstreams. This may require to encode for each channel of D information (e.g., transformation information 22) about how to calculate the weighted sum of the channels contained in the sub-bitstreams. For example, for each channel of D and for each of the channels contained in the sub-bitstreams (or one or more sets of the channels 14), one weight value may be encoded in the bitstream 12. Note, that the number of channels contained in the sub-bitstreams doesn't need to match the number of channels in D (e.g., K≠K′). In fact, it may be advantageous in terms of compression efficiency if the number of channels contained in the sub-bitstreams is smaller (e.g., K>K′) than the number of channels in D. Note that channels may have different sampling frequencies. If so, linear combinations may require a resampling of some of the channels contained in the sub-bitstreams to a common sampling frequency like, for example, the frequency of the channel to be reconstructed.

In a preferred embodiment, predefined sets of weight values for calculating the linear combinations are defined and it is indicated in the bitstream 12 (for example by using a flag or an index) whether one of these sets shall be used. This requires less bits than explicitly signaling all weights in the bitstream. For example, the weights of the inverse discrete cosine transform (IDCT) could be indicated by an information encoded in the bitstream 12 (for example by a flag or an index).

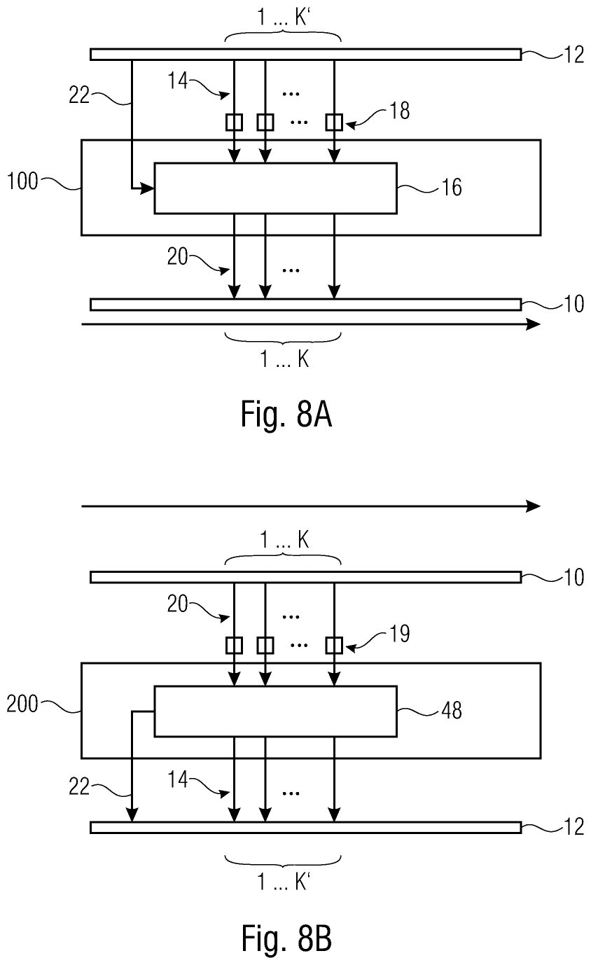

FIG. 8a shows a schematic view of a decoder 100 for decoding a multi-channel digital signal 10 (e.g., a biophysiological signal, e.g., any multi-channel digital signal 10 disclosed herein) from a data stream 12, configured to decode (see reference sign 29) K′ coded channels 14, which represent the multi-channel digital signal 10, from the data stream 12, and subject the K′ coded channels 14 to a channel re-transformation 16 which re-transforms co-aligned (e.g., temporally co-located) sample positions 18 of the K′ coded channels 14 so as to obtain K channels 20 of the multi-channel digital signal 10.

The decoder 100 shown in FIG. 8a may additionally comprise any feature (e.g., in isolation or in any combination) of any decoder 100 disclosed herein, e.g., with reference to other figures (e.g., FIGS. 2 and 4). Further features of the decoder 100 of FIG. 8a will in the following be described with reference to FIGS. 2 and 4, wherein it is noted that the use of an audio decoding scheme is optional. The description above for FIGS. 2 and 4 may partially or fully be applied to the decoder 100 shown in FIG. 8a.

The number K′ of decoded channels may be equal to the number K of obtained channels 20 (e.g., K=K′). The channel re-transformation 16 may be linear. The decoder 100 may be configured to derive the channel re-transformation 16 from transformation information 22 in the data stream 12.

The decoder 100 may be configured to update the channel re-transformation 16 based on the transformation information 22 in the data stream (e.g. at each RAP, e.g., so that different channel re-transformations are used before and after the updating). The decoder 100 may be configured to decode the K′ coded channels 14 of the multi-channel digital signal 10 from the data stream 12 using an audio decoding scheme (e.g., a multi-channel or spatial-object audio decoding scheme, e.g., any audio decoding scheme disclosed herein). The audio decoding scheme may be (or use) a multi-channel audio decoding scheme.

The decoder 100 may be configured to decode the K′ coded channels 14 of the multi-channel digital signal 10 from the data stream 12 using a decoding scheme which involves (e.g. reference sign 31 in FIG. 2) transform based decoding including deriving from the data stream 12 scale factors and transform coefficients, spectrally shaping the transform coefficients using the scale factors to obtain shaped spectra, re-transforming the shaped spectra to obtain frame signals and subjecting the frame signals to an overlap-add process (e.g., wherein the re-transformation may be an inverse MDCT), or linear predictive coding (LPC) decoding including deriving from the data stream 12 LPC coefficients and information on a residual signal and subjecting the residual signal to LPC synthesis by means of the LPC coefficients. The deriving of the scale factors from the data stream may involve entropy decoding the scale factors from the data stream 12 or decoding LPC coefficients from the data stream 12 and converting the LPC coefficients into the scale factors.

The decoder 100 may be configured to decode the K′ coded channels 14 of the multi-channel digital signal 10 from the data stream 12 using a multi-channel decoding scheme comprising decoding (see reference sign 31 in FIG. 2) an m-channel downmix signal 32, with 0<m<n, from the data stream 12 and deriving n coded channels 14, with n≤K′, by upmixing 33 the m-channel downmix signal 32 using side information 34 contained in the data stream 12. The decoder 100 may be configured to decode the K′ coded channels 14 of the multi-channel digital signal 10 from the data stream 12 by grouping 21 the K′ coded channels 14 into Q sets 25 of nᵢ coded channels 14, with nᵢ indicating the number of coded channels in set i, with Q≥i≥1 (e.g., based on channel grouping information 40 decoded from the data stream 12), and decoding at least one set j (e.g., each set j) of the Q sets 25, for which nⱼ≥2, from the data stream 12 using a multi-channel decoding scheme involving decoding an m-channel downmix signal 32, with m<nⱼ from the data stream 12 and deriving the nⱼ coded channels 14 of set j by upmixing the m-channel downmix signal 32 using side information 34 contained in the data stream 12 (e.g., wherein m may differ among the sets 35 coded using the multi-channel scheme; thus, notation mⱼ might then be used).

The multi-channel decoding scheme may be designed to maximally cope with a maximum number MAX of coded channels and the decoder 100 may be configured to group the K′ coded channels 14 into the Q sets 25 of nᵢ coded channels so that nᵢ≤MAX for each 0<i<Q+1. The side information 34 comprises one or more of ICC (inter-channel coherence) data, CLD (channel level difference) data, and CPC (channel prediction coefficient) data. The decoder 100 may be configured to decode channel grouping information 40 from the data stream 12 (e.g., as a basis for grouping 21 the K′ coded channels 14) and perform the grouping 21 using the channel grouping information 40. The channel grouping information 40 may comprise syntax elements indicating the number of coded channels nᵢ in the Q sets 25, and/or a sequential channel order among the K′ coded channels 14 using which the K′ coded channels 14 are grouped into the Q sets 25 so that coded channels 14 of different ones of the Q sets 25 are not interleaved along the sequential channel order and the one or more coded channels of one set immediately follow each other in the sequential channel order.

The decoder 100 may be configured to permute 50 the K channels 20 of the multi-channel digital signal 10 according to channel permutation information 52 signaled in the data stream 12 so as to obtain the multi-channel digital signal 10. The channel permutation information 52 may describe a permutation 50 of the N channels 20 obtained by the channel re-transformation 16 so as to result into a predetermined representation of the multi-channel digital signal 10. The multi-channel digital signal 10 may comprise (e.g., or be) a biophysiological signal 102 (e.g., any biophysiological signal 102 disclosed herein).

FIG. 8b shows a schematic view of an encoder 200 for encoding a multi-channel digital signal 10 into a data stream 12, configured to subject K channels 20 of the multi-channel digital signal 10 to a channel transformation 48 which transforms co-aligned (temporally co-located) sample positions 19 of the K channels 20 so as to obtain K′ coded channels 14 and encode the K′ coded channels 14 which represent the multi-channel digital signal 10 into the data stream 12.

The encoder 100 may be configured to re-sample one or more of the K channels 20 of the multi-channel digital signal 10 so as to render the K channels 20, or one or more sets of the K channels 20 mutually sampled at equal frequency and equal sampling phase, and/or render the K channels 20, or one or more sets of the K channels 20 mutually sampled at equal frequencies which are integer multiples of a common base frequency and phase adjusted

The encoder 200 shown in FIG. 8b may have one or more additional features of any encoder 200 disclosed herein. The encoder 200 shown in FIG. 8b may be configured to encode a data stream 12 decodable by the decoder 100 shown in FIG. 8a. The encoder 200 may comprise any encoder side features that corresponding to features of the decoder 100 shown in FIG. 8a.

### Multi-layer Linear Combinations of Decoded Channels

It may be advantageous if several linear transforms can be applied successively to sequence D (e.g., multi-channel digital signal 10). For example, a DCT could be applied to which typically compacts the signal energy to a small subset of the transformed channels (e.g., channels 14). Consequently, it may then be a good idea to apply a KLT only to such a subset of the transformed channels that have a high signal energy. The resulting signal may then, for example, consist of two sets of transformed channels (or any other number of channels). For example, one set of channels may be a result of applying the DCT followed by the KLT and another set of channels may be a result of applying the DCT only. Both sets (e.g., sets 25) can then be encoded into the bitstream (e.g., bit stream 12). The decoder operates in reversed order. For example, tt first applies the inverse KLT to the associated set of channels and then the inverse DCT over all channels in order to reconstruct (a potentially distorted version of) signal D.

In this invention, a bitstream (e.g., bit stream 12) may contain sub-bitstreams that were produced by involving several linear transforms (e.g., partial transformations 24) and the decoding of such a bitstream may be performed as follows. Let M be the number of linear combination stages that are successively applied in order to reconstruct the (potentially distorted) sequence D. Let Dᵢ₋₁ be the sequence of digital waveform data before applying the the i-th linear combination stage. I.e., D₀ is comprised of the channels as decoded from the sub-bitstreams (before applying the first linear combination stage, e.g., channels 14). D₁ is the result of applying the first linear combination stage and so on. Eventually, D_{M} is the result of applying the last linear combination stage and it corresponds to the (potentially distorted) reconstructed sequence D (e.g., channels 20). The number of channels of signals Dᵢ may be different for each i.

In a preferred embodiment, a linear combination stage i subdivides the channels of input signal Di-1 into two subsets. One subset is bypassed (e.g., performing an identity transformation, e.g., not using channels of said subset in stage i and instead as input at stage i+1) to the output of linear combination stage i while the other subset is converted by carrying out the linear combinations. Note that the linear combination stage can be interpreted as a 2D matrix multiplication. The bypassed channels then correspond to rows where only the diagonal element is 1 while all other values are 0. This technique reduces the number of weights required to calculate the linear combinations which may save weight signalling cost and computational complexity.

In another preferred embodiment, the weights associated with a linear combination stage are signalled explicitly or they are be chosen from a predefined set of transforms (indicating the chosen transform in the bitstream).

In another preferred embodiment, the first linear combination stage applies a first inverse transform (for example an inverse KLT) to a subset (or all) of the channels of D₀ and bypasses the remaining channels (if any) to D₁. The weights of the first inverse transform may be either explicitly signalled or chosen from a first set of transforms and indicated in the bitstream. The second linear combination stage may apply a second inverse transform (for example an inverse DCT) to a subset (or all) of the channels of D₁ and bypasses the remaining channels (if any) to D₂. The weights of the second inverse transform may be either explicitly signalled or chosen from a second set of transforms and indicated in the bitstream.

In another preferred embodiment, an offset value is associated with each weight value which is added to the outcome of the multiplication of the channel with the weight. Offset values may, for example, explicitly be signalled in the bitstream.

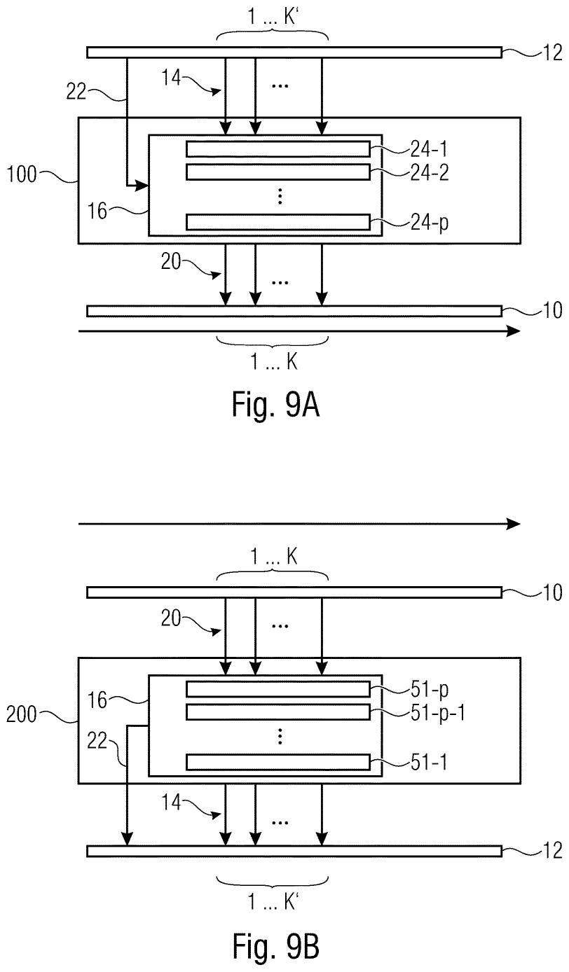

FIG. 9a shows a schematic view of the decoder 100 of FIG. 8a (but the principle may be applied to any decoder 100 disclosed herein), wherein the channel re-transformation 16 involves a sequence of partial channel re-transformations 24-1 to 24-p (summarized with reference sign 24). The partial channel re-transformations 24 may be of different dimensions in terms of number of re-transformed channels so that different coded channels are affected by different subsets (wherein each subset may be a proper subset of the overall set encmpoassing all (re-)transformations or equal to the overall set), or different numbers or sub-sequences of (re-)transformations, out of the sequence of the partial channel re-transformations 24.

FIG. 9b shows a schematic view of the encoder 200 of FIG. 8b (but the principle may be applied to any decoder 200 disclosed herein), wherein the channel transformation 48 involves a sequence of partial channel transformations 51-p to 51-1 (summarized with reference sign 51).

The partial channel transformations 51 may be of different dimensions in terms of number of transformed channels so that different coded channels are affected by different subsets (wherein each subset may be a proper subset of the overall set encmpoassing all (re-)transformations or equal to the overall set), or different numbers or sub-sequences of (re-)transformations, out of the sequence of the partial channel transformations 51.

### Combination of Inter-channel Delay With Linear Combinations of Decoded Channels

In order to achieve a good decorrelation of the signal by linear transforms in channel direction, it can be crucial (e.g., coding efficient) that the channels are exactly aligned. I.e., if there exists a delay between channels, linear combinations will likely fail in decorrelating the signal.

In this invention, a temporal delay value (e.g., in units of time and/or samples, e.g., buffer samples) can be applied individually to each channel (e.g., of all channels 20 or all channels 20 of a subset) in order to improve the decorrelation properties of the linear combination stages.

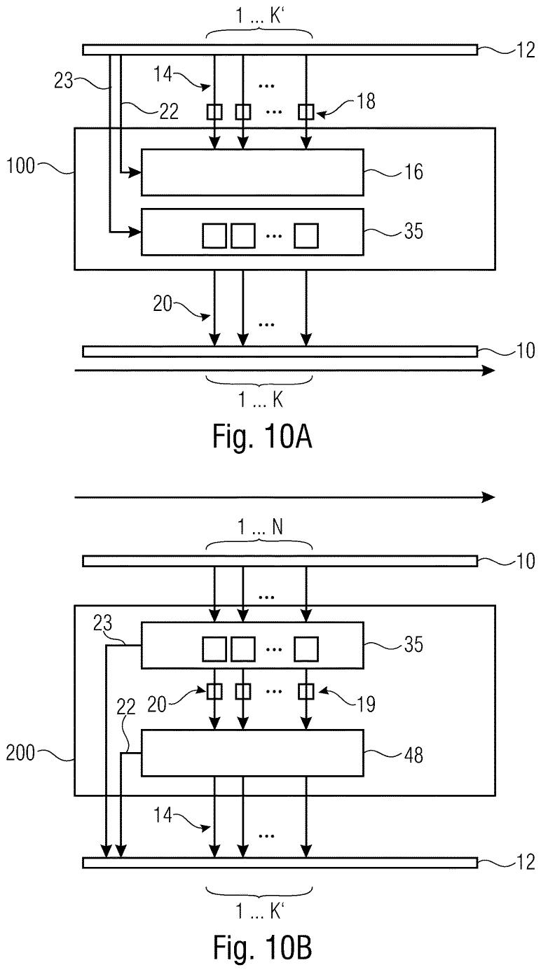

FIG. 10a shows a schematic view of the decoder 100 of FIG. 8a (but the principle may be applied to any decoder 100 disclosed herein, e.g., of FIG. 9a), wherein the decoder 100 is configured to decode inter-channel delay information 23 from the data stream 12 (further optionally, the K′ coded channels 14 of the multi-channel digital signal 10 might be decoded from the data stream 12 using inter-channel decoding dependencies, e.g., using inter-channel prediction), and mutually delay the K channels 20 according to the inter-channel delay information 23.

The decoder 100 may be configured to mutually delay 35 the K channels 20 according to the inter-channel delay information 23 in a manner so that the two of the K channels are subject to a mutual delay larger than one sample pitch (e.g., two, three, four, or more sample pitches) between the samples of the two channels.

FIG. 10b shows a schematic view of the encoder 200 of FIG. 8b (but the principle may be applied to any decoder 200 disclosed herein, e.g., encoder 100 of FIG. 9b), wherein the multi-channel digital signal has N channels (e.g., K channels 20) and the encoder 200 is configured to encode inter-channel delay information 23 into the data stream 12 (further optionally, the K′ coded channels 14 of the multi-channel digital signal 10 may be encoded into the data stream 12 using inter-channel decoding dependencies, e.g., using inter-channel prediction), and encode the N channels into the data stream 12 mutually delayed 35 according to the inter-channel delay information 23.

The multi-channel digital signal 10 may have N channels and the encoder 200 may be configured to encode the N channels into the data stream 12 mutually delayed 35 according to the inter-channel delay information 23 in a manner so that the two of the K channels 20 are subject to a mutual delay 35 larger than one sample pitch between the samples of the two channels.

### Bitstream Arrangement for Low Delay Coding

It may be of interest to arrange the bitstream (e.g., bit stream 12) in a way so that only sample values up to a predefined instant of time t₁ (e.g., measured in time and/or samples) are required to produce a decodable portion of the bitstream that allows for a reconstruction of samples in sequence D (e.g., multi-channel digital signal 10) up to a predefined instant of time t₂≤t₁. The difference Δ=t₁−t₂ (e.g., temporal intervals 26) is denoted coding delay associated with the portion. In a communication scenario, it may be of interest to arrange the bitstream in a way to guarantee that a particular coding delay is not exceeded by any of the bitstream portions.

Assume, a sub-bitstreams as produced using the target codec can be arranged in portions (e.g., substream portions 30) that can be decoded in sequence so that each decoded portion may produce some reconstructed sample values that may be employed when reconstructing (the potentially distorted) sequence D.

In this invention, the bitstream can be arranged in ordered portions (e.g., substream portions 30) that can be decoded in sequence. Whenever the encoder has produced a new portion, it can be decoded by the decoder. Let pₙ be the n-th portion of the bitstream, \hat{t}ₙ be the instant in time up to which sample values of sequence D are required in order to produce portion pₙ, and let t̅ₙ be the instant in time up to which (a potentially distorted versions of) sample values of D can be reconstructed from decoding portion pₙ.

In a preferred embodiment, each bitstream portion pₙ is arranged in a way so that the associated delay Δₙ=\hat{t}ₙ−t̅ₙ (e.g., temporal interval 26 or a delay based thereon) does not exceed a predetermined maximum delay value.

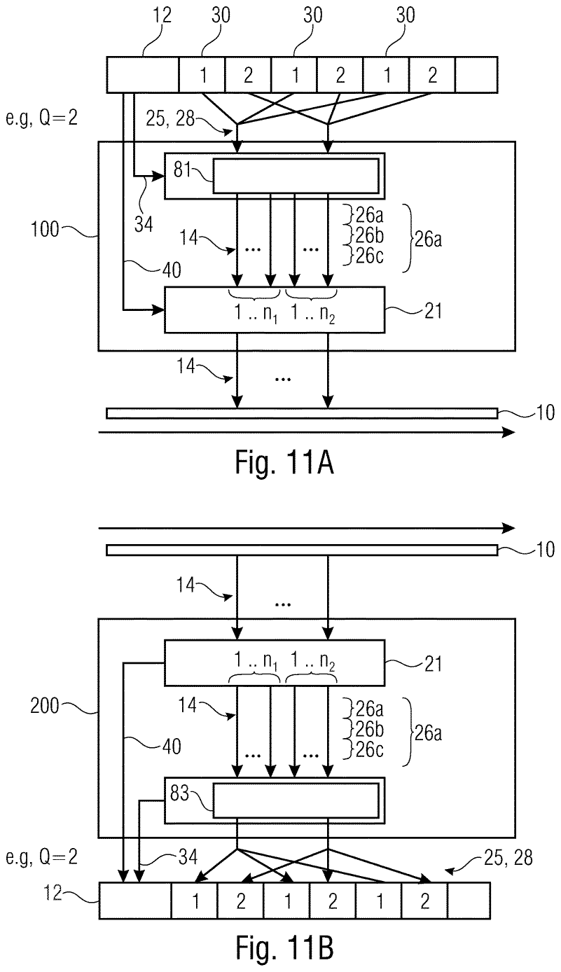

FIG. 11a shows the decoder 100 of FIG. 1a (or FIG. 5a), wherein the biophysiological signal 102 (or any other type of signal disclosed herein such as an audio signal or seismic data) is a multi-channel digital signal 10, and the decoding the multi-channel digital signal 10 from the data stream 12 using the audio decoding scheme (or a decoding scheme with a similar functionality), comprises grouping 21 K′ coded channels 14 representing the multi-channel digital signal 10 into Q sets 25 of nᵢ coded channels, with nᵢ indicating the number of coded channels in set i, with Q≥i≥1 (e.g., based on channel grouping information 40 decoded from the data stream 12), and decoding at least one set j of the Q sets 25, for which nⱼ≥2, from the data stream 12 using the audio decoding scheme with the audio decoding scheme being a multi-channel audio decoding scheme involving decoding an m-channel downmix signal 32, with m<nⱼ from the data stream 12 and deriving the nⱼ coded channels 14 of set j by upmixing the m-channel downmix signal 32 using side information 34 contained in the data stream 12.

The decoder 100 may be further configured to decode each set 25 of the Q sets from a substream 28 of the data stream 12 which is associated with the respective set 25 and in units of temporal intervals 26 (e.g., intervals 26a, b, c) so that each substream 28 is formed by a sequence of substream portions 30 (wherein in FIG. 11a only the substream portions 30 with the label “1” are depicted with the reference sign 30 to better visualize that they relate to the same set, but substream portion 30 with the label “2” are also substream portions 30) having consecutive temporal intervals of the set 25 associated with the respective substream 28 encoded thereinto, and the substream portions 30 of the substreams 28 are mutually interleaved (e.g., coded in an alternating manner) so that substream portions 30 whose temporal interval overlaps are immediately consecutive in the data stream 12 and precede substream portions 30 whose temporal interval temporally follows.

Similarly, the decoder of FIG. 5 a may be configured to decode each set 25 of the Q sets 25 from a substream 28 of the data stream 12 which is associated with the respective set 25 and in units of temporal intervals 26 wherein each substream 28 is formed by a sequence of substream portions 30 having consecutive temporal intervals 26 of the set 25 associated with the respective substream 28 encoded thereinto, and the substream portions 30 of the substreams are mutually interleaved so that substream portions 30 whose temporal interval 26 overlaps are immediately consecutive in the data stream 12 and precede substream portions 30 whose temporal interval 26 temporally follows. However, the same principle may be applicable to any other decoder 100 disclosed herein.

FIG. 11b shows the encoder 200 of FIG. 1b (or FIG. 5b), wherein the biophysiological signal (or any other type of signal disclosed herein such as an audio signal or seismic data) is a multi-channel digital signal 10, and the encoding the multi-channel digital signal 10 into the data stream 12 using the audio encoding scheme, comprises grouping K′ coded channels 14 representing the multi-channel digital signal 10 into Q sets 25 of nᵢ coded channels 14, with nᵢ indicating the number of coded channels 14 in set i, with Q≥i≥1 (e.g., wherein the grouping may be derivable based on channel grouping information 40 encoded into the data stream 12), and encoding at least one set j of the Q sets, for which nⱼ≥2, into the data stream 12 using the audio encoding scheme 83 with the audio encoding scheme being a multi-channel audio encoding scheme involving encoding an m-channel downmix signal 32, with m<nⱼ, into the data stream 12 which allows deriving the nⱼ coded channels 14 of set j by upmixing the m-channel downmix signal 32 using the side information 34 contained in the data stream.

The encoder 200 may be configured to encode each set of the Q sets 25 into a substream 28 of the data stream 23 which is associated with the respective set and in units of temporal intervals 26 so that each substream 28 is formed by a sequence of substream portions 30 having consecutive temporal intervals 26 of the set 25 associated with the respective substream 28 encoded thereinto, and the substream portions 30 of the substreams 28 are mutually interleaved so that substream portions 30 whose temporal interval 26 overlaps are immediately consecutive in the data stream 12 and precede substream portions 30 whose temporal interval 26 temporally follows.

Similarly, the decoder of FIG. 5 b may be configured to encode each set 25 of the Q sets 25 into a substream 28 of the data stream 12 which is associated with the respective set 25 and in units of temporal intervals 26 wherein each substream 28 is formed by a sequence of substream portions 30 having consecutive temporal intervals 26 of the set associated with the respective substream 28 encoded thereinto, and the substream portions 30 of the substreams 28 are mutually interleaved so that substream portions 30 whose temporal interval 26 overlaps are immediately consecutive in the data 12 stream and precede substream portions 30 whose temporal interval 26 temporally follows. However, the same principle may be applicable to any other encoder 200 disclosed herein.

The time intervals 26 may have an identical length, e.g., in terms of numbers of samples or time duration. The time intervals 26 may be (or may be based on) the sample length of temporal blocks 65 in FIG. 4 (e.g., with 16 samples 63, or any other number of samples 63).

### Bitstream Arrangement for Random Access Decoding

It may be of interest to arrange the bitstream (e.g., bit stream 10) in a way so that it supports random access decoding. I.e., the decoder (e.g., decoder 100) can identify a position in the bitstream that supports random access decoding and start decoding from there.

Assume that any portion of a sub-bitstream (e.g., temporal blocks 65 as shown in FIG. 4, e.g., separated by borders 96 and/or borders next to access coded channels 88a and 88b in FIG. 4) can be arranged in a way so that it allows for starting decoding from there.

In this invention, the encoder (e.g., encoder 200) may produce sub-bitstreams by employing the target codec in a way so that they can be arranged in portions and that some of the portions can be used to start decoding from them.

In a preferred embodiment, the bitstream is arranged in portions (e.g., temporal blocks 65) of which some support random access decoding (e.g., temporal blocks 65 separated by borders 96).

In another preferred embodiment, the bitstream is arranged in a way so that the following decoding procedure can be employed. The decoder starts decoding of the bitstream at a random access position in the bitstream (e.g., temporal blocks 65b and 65e in FIG. 4). It extracts the portions of sub-bitstreams and identifies the contained random access positions. Then it starts decoding of the sub-bitstreams at the random access positions. This produces decoded sequences of samples for different channels that may start at different instants of time. I.e., for some instants of time, there may be decoded samples for some of the channels while there are no decoded samples for other channels. Consequently, the decoder identifies the first instant in time for which all channels have produced decoded samples. This is the instant in time at which the random access decoding starts.

In another preferred embodiment, a bitstream portion that supports random access also allows zero, one, or more layers of linear combinations and/or an optional subsequent inter-channel delay adjustment when reconstructing the signal.

For example, for the decoder 100 of FIG. 11a, in each substream 28, some of the sequence of substream portions 30 may be coded as random access points. However, the same principle may be applied to any other decoder 100 disclosed herein (e.g., decoders 100 configured to decode each set of Q sets from a substream 28 of the data stream 12).

The random access points of the substreams 28 may be temporally aligned. Alternatively, the random access points of the substreams 28 may not be temporally aligned.

In each substream 28, some of the sequence of substream portions 30 may be coded as random access points (e.g., substream portions 30 that relate to temporal blocks 65b, 65e in FIG. 4).

Similarly, for the encoder 200 of FIG. 11b, for in each substream 28, some of the sequence of substream portions 30 may be coded as random access points. However, the same principle may be applied to any other encoder 200 disclosed herein (e.g., encoders 200 configured to encode each set of Q sets 25 into a substream 28 of the data stream 12).

### Support for Channels of Different Sampling Rates and/or Types

As noted earlier, in some instances of D (e.g., multi-channel digital signal 10), different sampling rates may be used in different channel signals (e.g., in channels 14 and/or 20). Moreover, some signals may be bandwidth limited to such an extent that encoding, and transmission in the bitstream, in a downsampled representation may be feasible without significant increase in distortion during encoding. Finally, some channel signals may not actually be biomedical signals but human-readable annotation text, possibly sparse and temporally aligned to some peculiarities in one or more other (biomedical) channel signals in D, or some even further types of non-waveform signal data. Similarly, the multi-channel digital signal 10 comprising different type of data such as seismic measurement data or weather data is described herein may be used.

To account for such different possible characteristics of the signals in D, it is proposed to allow the usage of different coding technologies, or in other words, codec specifications, in dependence of the individual signal characteristics of the channel signals in D. A preferred embodiment (e.g., primarily focused on encoding but may similarly be applied for decoding) may, therefore, appear as follows.
1) typical biomedical (or other data source) waveform data: clustering (e.g., grouping according to channels having or not having periodic and/or waveform type characteristics, e.g., grouping based on one or more criteria and/or a predefined grouping, e.g., according to data source input) of the respective channel signals (e.g., channels 20) of this type according to any of the previously described embodiments, followed by usage of a (time-and/or transform-domain) waveform coding technology such as AAC, HE-AAC (High Efficiency Advanced Audio Coding), xHE-AAC, or some other lossless or lossy waveform codec.
2) band-limited waveform data: clustering (e.g., grouping 44 in FIG. 3, e.g., grouping according to similarity of one or more characteristics comprising one or more of amplitude, frequencies, frequency spectrums, and offset) of the respective channel signals of this type according to any of the previously described embodiments, followed by downsampling (e.g., by a factor of 2 or any other factor) and usage of waveform coding technology. To undo the downsampling, the decoded channel signals are upsampled (e.g., by a factor of 2) after decoding by the employed waveform codec. Note that said waveform codec may not necessarily have to be identical to the one used in case 1). In fact, when using the same codec on downsampled channel signals, the codec's frames may span a longer time period (e.g., twice the time period when using an AAC codec flavor and downsampling by 2). To synchronize the framing between band-limited and non-band-limited channel signal coding, it may therefore be desirable to, in case 2), use a waveform codec having a shorter- and preferably, proportional-frame size than the waveform codec used in case 1). To give a general example not intended as limiting the scope of this embodiment to the AAC family of codecs, AAC-LD employs a shorter frame size than AAC or (x)HE-AAC.
3) textual, e.g. human-readable, data: such (often temporally sparse) data may be most efficiently encoded, preferably but not necessarily in a lossless manner, via, e.g., run-length encoding and/or “zip” technology such as the Lempel-Ziv or Lempel-Ziv-Welch algorithm, as opposed to a waveform targeting codec.

### Implementations as Methods and Data Streams

Further is provided a method performed by any decoder 100 disclosed herein. For example, a method is provided for decoding a biophysiological signal 102 from a data stream 12, the method comprising decoding the biophysiological signal 102 from the data stream 12 using an audio decoding scheme (e.g., performable by the decoder 100 shown in FIG. 1a). Similarly, methods are provided that are performable by any decoder 100 disclosed herein, such as shown in FIGS. 2, 4, 5a, 6a, 7a, 8a, 9a, 10a, and 11a.

Further is provided a method performed by any encoder 100 disclosed herein. For example, a method is provided for encoding a biophysiological signal 102 into a data stream 12, the method comprising encoding the biophysiological signal 102 into the data stream 12 using an audio coding scheme (e.g., performable by the decoder 100 shown in FIG. 1b). Similarly, methods are provided that are performable by any encoder 200 disclosed herein, such as shown in FIGS. 3, 4, 5b, 6b, 7b, 8b, 9b, 10b, and 11b.

Further is provided a bit stream 12 (or data stream or data stream comprising the bit stream 12) encoded by any of the encoder 200 (or a method performed by such an encoder 200) as disclosed herein. The bit stream 12 may be decodeable by any of the decoders 100 (or a method performed by such a decoder 100) as disclosed herein. The bit stream 12 may be stored on a digital storage medium, e.g., a non-transitory storage medium.

Also, further embodiments will be defined by the enclosed claims.

It should be noted that any embodiments as defined by the claims can be supplemented by any of the details (features and functionalities) described in the above mentioned chapters.

Also, the embodiments described in the above mentioned chapters can be used individually, and can also be supplemented by any of the features in another chapter, or by any feature included in the claims.

Also, it should be noted that individual aspects described herein can be used individually or in combination. Thus, details can be added to each of said individual aspects without adding details to another one of said aspects.

Moreover, features and functionalities disclosed herein relating to a method can also be used in an apparatus (configured to perform such functionality). Furthermore, any features and functionalities disclosed herein with respect to an apparatus can also be used in a corresponding method. In other words, the methods disclosed herein can be supplemented by any of the features and functionalities described with respect to the apparatuses.

Also, any of the features and functionalities described herein can be implemented in hardware or in software, or using a combination of hardware and software, as will be described in the section “implementation alternatives”.

Also, any of the features and functionalities described herein can be implemented in hardware or in software, or using a combination of hardware and software, as will be described in the section “implementation alternatives”.

### Implementation Alternatives

Although some aspects have been described in the context of an apparatus (e.g., encoder), it is clear that these aspects also represent a description of the corresponding method, where a block or device corresponds to a method step or a feature of a method step. Analogously, aspects described in the context of a method step also represent a description of a corresponding block or item or feature of a corresponding apparatus. Some or all of the method steps may be executed by (or using) a hardware apparatus, like for example, a microprocessor, a programmable computer or an electronic circuit. In some embodiments, one or more of the most important method steps may be executed by such an apparatus.

Depending on certain implementation requirements, embodiments of the invention can be implemented in hardware or in software. The implementation can be performed using a digital storage medium, for example a floppy disk, a DVD, a Blu-Ray, a CD, a ROM, a PROM, an EPROM, an EEPROM or a FLASH memory, having electronically readable control signals stored thereon, which cooperate (or are capable of cooperating) with a programmable computer system such that the respective method is performed. Therefore, the digital storage medium may be computer readable. The encoded media signal may be encoded into a data stream. The data stream may be stored on a digital storage medium as described above (e.g., a transitory digital storage medium).

Some embodiments according to the invention comprise a data carrier having electronically readable control signals, which are capable of cooperating with a programmable computer system, such that one of the methods described herein is performed.

Generally, embodiments of the present invention can be implemented as a computer program product with a program code, the program code being operative for performing one of the methods when the computer program product runs on a computer. The program code may for example be stored on a machine readable carrier (e.g., non-transitory storage medium).

Other embodiments comprise the computer program for performing one of the methods described herein, stored on a machine readable carrier.

In other words, an embodiment of the inventive method is, therefore, a computer program having a program code for performing one of the methods described herein, when the computer program runs on a computer.

A further embodiment of the inventive methods is, therefore, a data carrier (or a digital storage medium, or a computer-readable medium) comprising, recorded thereon, the computer program for performing one of the methods described herein. The data carrier, the digital storage medium or the recorded medium are typically tangible and/or non-transitionary.

A further embodiment of the inventive method is, therefore, a data stream or a sequence of signals representing the computer program for performing one of the methods described herein. The data stream or the sequence of signals may for example be configured to be transferred via a data communication connection, for example via the Internet.

A further embodiment comprises a processing means, for example a computer, or a programmable logic device, configured to or adapted to perform one of the methods described herein.

A further embodiment comprises a computer having installed thereon the computer program for performing one of the methods described herein.

A further embodiment according to the invention comprises an apparatus or a system configured to transfer (for example, electronically or optically) a computer program for performing one of the methods described herein to a receiver. The receiver may, for example, be a computer, a mobile device, a memory device or the like. The apparatus or system may, for example, comprise a file server for transferring the computer program to the receiver.

In some embodiments, a programmable logic device (for example a field programmable gate array) may be used to perform some or all of the functionalities of the methods described herein. In some embodiments, a field programmable gate array may cooperate with a microprocessor in order to perform one of the methods described herein. Generally, the methods are preferably performed by any hardware apparatus.

The apparatus described herein may be implemented using a hardware apparatus, or using a computer, or using a combination of a hardware apparatus and a computer.

The apparatus described herein, or any components of the apparatus described herein, may be implemented at least partially in hardware and/or in software.

The methods described herein may be performed using a hardware apparatus, or using a computer, or using a combination of a hardware apparatus and a computer.

The methods described herein, or any components of the apparatus described herein, may be performed at least partially by hardware and/or by software.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations and equivalents as fall within the true spirit and scope of the present invention.

1.-43. (canceled)

43. Decoder for decoding a multi-channel digital signal from a data stream, configured to
decode channel grouping information from the data stream, and
group K′ coded channels representing the multi-channel digital signal into Q sets of nᵢ coded channels, with nᵢ indicating the number of coded channels in set i, with Q≥i≥1, according to the channel grouping information, and
decode at least one set j of the Q sets, for which nⱼ≥2, from the data stream using a multi-channel decoding scheme.

44. Decoder according to claim 43, wherein
the channel grouping information comprises
syntax elements indicating the number of coded channels nᵢ in the Q sets, and/or
a sequential channel order among the K′ coded channels using which the K′ coded channels are grouped into the Q sets so that coded channels of different ones of the Q sets are not interleaved along the sequential channel order and the one or more coded channels of one set immediately follow each other in the sequential channel order.

45. Decoder according to claim 43, configured to
decode each set j of the Q sets, for which nⱼ≥2, from the data stream using the multi-channel decoding scheme.

46. Decoder according to claim 43, wherein
the multi-channel decoding scheme involves decoding an m-channel downmix signal, with m<nⱼ from the data stream and deriving the nⱼ coded channels by upmixing the m-channel downmix signal using side information contained in the data stream to obtain the nⱼ coded channels.

47. Decoder according to claim 46, wherein the side information comprises one or more of
inter-channel coherence, ICC, data,
channel level difference, CLD, data, and
channel prediction coefficient, CPC, data.

48. Decoder according to claim 43, wherein
the multi-channel decoding scheme is designed to maximally cope with a maximum number MAX of coded channels and the decoder is configured to group the K′ coded channels representing the multi-channel digital signal into the Q sets of nᵢ coded channels so that nᵢ≤MAX for each 0<i<Q+1.

49. Decoder according to claim 43, configured to
subject the K′ coded channels representing the multi-channel digital signal to a channel re-transformation which re-transforms co-aligned sample positions of the K′ coded channels so as to obtain K channels of the multi-channel digital signal.

50. Decoder according to claim 49, configured to derive the channel re-transformation from transformation information in the data stream.

51. Decoder according to claim 50, configured to update the channel re-transformation based on the transformation information in the data stream.

52. Decoder according to claim 49, wherein the channel re-transformation involves a sequence of partial channel re-transformations.

53. Decoder according to claim 52, wherein
the partial channel re-transformations are of different dimensions in terms of number of re-transformed channels so that different coded channels are affected by different subsets, or different numbers or sub-sequences of transformations, out of the sequence of the partial channel re-transformations.

54. Decoder according to claim 43, wherein
the multi-channel decoding scheme is a multi-channel audio decoding scheme.

55. Decoder according to claim 43,
wherein the multi-channel decoding scheme involves
transform based decoding including deriving from the data stream scale factors and transform coefficients, spectrally shaping the transform coefficients using the scale factors to obtain shaped spectra, re-transforming the shaped spectra to obtain frame signals and subjecting the frame signals to an overlap-add process, or
linear predictive coding, LPC, decoding including deriving from the data stream LPC coefficients and information on a residual signal and subjecting the residual signal to LPC synthesis by means of the LPC coefficients.

56. Decoder according to claim 55, wherein
the deriving of the scale factors from the data stream involves entropy decoding the scale factors from the data stream or decoding LPC coefficients from the data stream and converting the LPC coefficients into the scale factors.

57. Decoder according to claim 43, wherein the multi-channel digital signal has K channels and the decoder is configured to
decode inter-channel delay information from the data stream, and
mutually delay the K channels according to the inter-channel delay information.

58. Decoder according to claim 43,
wherein the multi-channel digital signal comprises a biophysiological signal.

59. Decoder according to claim 43, configured to
permute the channels of the multi-channel digital signal according to channel permutation information signaled in the data stream so as to obtain the multi-channel digital signal.

60. Decoder according to claim 59, wherein the channel permutation information describes a permutation of the channels so as to result into a predetermined representation of the multi-channel digital signal.

61. Decoder according to claim 43, configured to
decoding each set of the Q sets from a substream of the data stream which is associated with the respective set and in units of temporal intervals wherein each substream is formed by a sequence of substream portions having consecutive temporal intervals of the set associated with the respective substream encoded thereinto, and
the substream portions of the substreams are mutually interleaved so that substream portions whose temporal interval overlaps are immediately consecutive in the data stream and precede substream portions whose temporal interval temporally follows.

62. Decoder according to claim 61, wherein
in each substream, some of the sequence of substream portions are coded as random access points.

63. Decoder according to claim 62, wherein
the random access points of the substreams are temporally aligned, or
the random access points of the substreams are not temporally aligned.

64. (canceled)

65. Encoder for encoding a multi-channel digital signal into a data stream, configured to
encode channel grouping information into the data stream, using which K′ coded channels representing the multi-channel digital signal are grouped into Q sets of nᵢ coded channels, with nᵢ indicating the number of coded channels in set i, with Q≥i≥1, and
encode at least one set j of the Q sets, for which nⱼ≥2, into the data stream using a multi-channel coding scheme.

66.-230. (canceled)

231. Method for decoding a multi-channel digital signal from a data stream, the method comprising
decoding channel grouping information from the data stream, and
grouping K′ coded channels representing the multi-channel digital signal into Q sets of nᵢ coded channels, with nᵢ indicating the number of coded channels in set i, with Q≥i≥1, according to the channel grouping information, and decoding at least one set j of the Q sets, for which nⱼ≥2, from the data stream using a multi-channel decoding scheme.

232.-235. (canceled)

236. Method for encoding a multi-channel digital signal into a data stream, the method comprising Q≥i≥1, encoding channel grouping information into the data stream, using which K′ coded channels representing the multi-channel digital signal are grouped into Q sets of nᵢ coded channels, with nᵢ indicating the number of coded channels in set i, with Q≥i≥1, and Q≥i≥1, encoding at least one set j of the Q sets, for which nⱼ≥2, into the data stream using a multi-channel coding scheme.

237.-240. (canceled)

241. Data stream generated using an encoding method according to claim 236.

242.-244. (canceled)
